SEQUENCE LISTING (382 sequences)

SEQ ID NO: 1 - protein, 15 aa, Homo sapiens
FEGTEMWNPNRELSE

SEQ ID NO: 2 - protein, 15 aa, Homo sapiens
FEGTEMWYPNRELSE

SEQ ID NO: 3 - protein, 15 aa, Homo sapiens
AVKRLPLVYCDYHGH

SEQ ID NO: 4 - protein, 15 aa, Homo sapiens
AVKRLPLIYCDYHGH

SEQ ID NO: 5 - protein, 15 aa, Homo sapiens
TCSCQSSGTSSTSYS

SEQ ID NO: 6 - protein, 15 aa, Homo sapiens
TCSCQSSWTSSTSYS

SEQ ID NO: 7 - protein, 15 aa, Homo sapiens
PWRKFPVYVLGQFLG

SEQ ID NO: 8 - protein, 15 aa, Homo sapiens
PWRKFPVHVLGQFLG

SEQ ID NO: 9 - protein, 15 aa, Homo sapiens
MNAAVTFANCALGRV

SEQ ID NO: 10 - protein, 15 aa, Homo sapiens
MNAAVTFTNCALGRV

SEQ ID NO: 11 - protein, 15 aa, Homo sapiens
INCLSSPNEETVLSA

SEQ ID NO: 12 - protein, 15 aa, Homo sapiens
INCLSSPSEETVLSA

SEQ ID NO: 13 - protein, 15 aa, Homo sapiens
STAYPAPMRRRCCLP

SEQ ID NO: 14 - protein, 15 aa, Homo sapiens
STAYPAPVRRRCCLP

SEQ ID NO: 15 - protein, 15 aa, Homo sapiens
VALKPQERVEKRQTP

SEQ ID NO: 16 - protein, 15 aa, Homo sapiens
VALKPQECVEKRQTP

SEQ ID NO: 17 - protein, 15 aa, Homo sapiens
PSHQPPASTLSPNPT

SEQ ID NO: 18 - protein, 15 aa, Homo sapiens
PSHQPPARTLSPNPT

SEQ ID NO: 19 - protein, 15 aa, Homo sapiens
TPEPAIPPKATLWPA

SEQ ID NO: 20 - protein, 15 aa, Homo sapiens
TPEPAIPHKATLWPA

SEQ ID NO: 21 - protein, 13 aa, Homo sapiens
MFTLTGCRLVEKT

SEQ ID NO: 22 - protein, 13 aa, Homo sapiens
MFTLTSCRLVEKT

SEQ ID NO: 23 - protein, 15 aa, Homo sapiens
THRPGGKHGRLAGGS

SEQ ID NO: 24 - protein, 15 aa, Homo sapiens
THRPGGKRGRLAGGS

SEQ ID NO: 25 - protein, 15 aa, Homo sapiens
VTVHPTSNSTATSQG

SEQ ID NO: 26 - protein, 15 aa, Homo sapiens
VTVHPTSKSTATSQG

SEQ ID NO: 27 - protein, 15 aa, Homo sapiens
STATHSPATTSHGNA

SEQ ID NO: 28 - protein, 15 aa, Homo sapiens
STATHSPSTTSHGNA

SEQ ID NO: 29 - protein, 15 aa, Homo sapiens
LQREYASVKEENERL

SEQ ID NO: 30 - protein, 15 aa, Homo sapiens
LQREYASMKEENERL

SEQ ID NO: 31 - protein, 13 aa, Homo sapiens
MEVSGCPTPAGQS

SEQ ID NO: 32 - protein, 13 aa, Homo sapiens
MEVSGWPTPAGQS

SEQ ID NO: 33 - protein, 15 aa, Homo sapiens
ARAAAAAAFEIDPRS

SEQ ID NO: 34 - protein, 15 aa, Homo sapiens
ARAAAAATFEIDPRS

SEQ ID NO: 35 - protein, 15 aa, Homo sapiens
HGLSHSLRQISSQLS

SEQ ID NO: 36 - protein, 15 aa, Homo sapiens
HGLSHSLWQISSQLS

SEQ ID NO: 37 - protein, 15 aa, Homo sapiens
VTTKKTPPSQPPGNV

SEQ ID NO: 38 - protein, 15 aa, Homo sapiens
VTTKKTPSSQPPGNV

SEQ ID NO: 39 - protein, 15 aa, Homo sapiens
SSLPGPPGPPGPPGP

SEQ ID NO: 40 - protein, 15 aa, Homo sapiens
SSLPGPPGPPGPRGY

SEQ ID NO: 41 - protein, 15 aa, Homo sapiens
FSISQLQKNHDMNDE

SEQ ID NO: 42 - protein, 15 aa, Homo sapiens
FSISQLQTNHDMNDE

SEQ ID NO: 43 - protein, 15 aa, Homo sapiens
GINQTTGALYLRVDS

SEQ ID NO: 44 - protein, 15 aa, Homo sapiens
GINQTTGTLYLRVDS

SEQ ID NO: 45 - protein, 14 aa, Homo sapiens
MSYDYHQNWGRDGG

SEQ ID NO: 46 - protein, 14 aa, Homo sapiens
MSYDYHHNWGRDGG

SEQ ID NO: 47 - protein, 15 aa, Homo sapiens
EMQEERLKLPILSEE

SEQ ID NO: 48 - protein, 15 aa, Homo sapiens
EMQEERLTLPILSEE

SEQ ID NO: 49 - protein, 15 aa, Homo sapiens
ENKNQELRSLISQYQ

SEQ ID NO: 50 - protein, 15 aa, Homo sapiens
ENKNQELHSLISQYQ

SEQ ID NO: 51 - protein, 15 aa, Homo sapiens
SSAEPTEHGERTPLA

SEQ ID NO: 52 - protein, 15 aa, Homo sapiens
SSAEPTENGERTPLA

SEQ ID NO: 53 - protein, 15 aa, Homo sapiens
VLACGLSRIWGEERG

SEQ ID NO: 54 - protein, 15 aa, Homo sapiens
VLACGLSQIWGEERG

SEQ ID NO: 55 - protein, 15 aa, Homo sapiens
LADGEGGGTDEGIYD

SEQ ID NO: 56 - protein, 15 aa, Homo sapiens
LADGEGGATDEGIYD

SEQ ID NO: 57 - protein, 15 aa, Homo sapiens
YVVPPPARPCPTSGP

SEQ ID NO: 58 - protein, 15 aa, Homo sapiens
YVVPPPAWPCPTSGP

SEQ ID NO: 59 - protein, 9 aa, Homo sapiens
MRETSGFTL

SEQ ID NO: 60 - protein, 9 aa, Homo sapiens
MRDKWLHIE

SEQ ID NO: 61 - protein, 15 aa, Homo sapiens
ECSECGKVFLESAAL

SEQ ID NO: 62 - protein, 15 aa, Homo sapiens
ECSECGKDFLESAAL

SEQ ID NO: 63 - protein, 15 aa, Homo sapiens
LTDHRAHRCPGDGDD

SEQ ID NO: 64 - protein, 15 aa, Homo sapiens
LTDHRAHCCPGDGDD

SEQ ID NO: 65 - protein, 15 aa, Homo sapiens
LTDHRAHRCPGGNAK

SEQ ID NO: 66 - protein, 15 aa, Homo sapiens
LTDHRAHCCPGGNAK

SEQ ID NO: 67 - protein, 15 aa, Homo sapiens
DEVSMKGRPPPTPLF

SEQ ID NO: 68 - protein, 15 aa, Homo sapiens
DEVSMKGGPPPTPLF

SEQ ID NO: 69 - protein, 15 aa, Homo sapiens
AQGWSTVARFQITAT

SEQ ID NO: 70 - protein, 15 aa, Homo sapiens
AQGWSTVSRFQITAT

SEQ ID NO: 71 - protein, 15 aa, Homo sapiens
KLVVVGAGGVGKSAL

SEQ ID NO: 72 - protein, 15 aa, Homo sapiens
KLVVVGAVGVGKSAL

SEQ ID NO: 73 - protein, 15 aa, Homo sapiens
LFGLGKDEGWGPPAR

SEQ ID NO: 74 - protein, 15 aa, Homo sapiens
LFGLGKDVGWGPPAR

SEQ ID NO: 75 - protein, 15 aa, Homo sapiens
VMMHGGPPHPGMPMS

SEQ ID NO: 76 - protein, 15 aa, Homo sapiens
VMMHGGPAHPGMPMS

SEQ ID NO: 77 - protein, 8 aa, Homo sapiens
MRHFCLIS

SEQ ID NO: 78 - protein, 9 aa, Homo sapiens
MHHFCLISE

SEQ ID NO: 79 - protein, 15 aa, Homo sapiens
PMEKPTISTEKPTIP

SEQ ID NO: 80 - protein, 15 aa, Homo sapiens
PMEKPTITTEKPTIP

SEQ ID NO: 81 - protein, 15 aa, Homo sapiens
YVSMMCNEQAYSLAV

SEQ ID NO: 82 - protein, 15 aa, Homo sapiens
YVSMMCNKQAYSLAV

SEQ ID NO: 83 - protein, 15 aa, Homo sapiens
LWTEGMLQMAFHILA

SEQ ID NO: 84 - protein, 15 aa, Homo sapiens
LWTEGMLKMAFHILA

SEQ ID NO: 85 - protein, 15 aa, Homo sapiens
KPVILGVRWYVETTS

SEQ ID NO: 86 - protein, 15 aa, Homo sapiens
KPVILGVCWYVETTS

SEQ ID NO: 87 - protein, 10 aa, Homo sapiens
TMLARLVSDS

SEQ ID NO: 88 - protein, 10 aa, Homo sapiens
TMLARLVLDS

SEQ ID NO: 89 - protein, 15 aa, Homo sapiens
SSGGGSSGGGYGGGS

SEQ ID NO: 90 - protein, 15 aa, Homo sapiens
SSGGGSSSGGYGGGS

SEQ ID NO: 91 - protein, 15 aa, Homo sapiens
DPSAIGLADPPIPSP

SEQ ID NO: 92 - protein, 15 aa, Homo sapiens
DPSAIGLVDPPIPSP

SEQ ID NO: 93 - protein, 15 aa, Homo sapiens
QLTAHKMIHTGEKPY

SEQ ID NO: 94 - protein, 15 aa, Homo sapiens
QLTAHKMNHTGEKPY

SEQ ID NO: 95 - protein, 15 aa, Homo sapiens
AVYTPPSVSTHQMPR

SEQ ID NO: 96 - protein, 15 aa, Homo sapiens
AVYTPPSDSTHQMPR

SEQ ID NO: 97 - protein, 15 aa, Homo sapiens
PGSGPQNPPGLGSGA

SEQ ID NO: 98 - protein, 15 aa, Homo sapiens
PGSGPQNAPGLGSGA

SEQ ID NO: 99 - protein, 15 aa, Homo sapiens
FASPGDDGDGRAEGF

SEQ ID NO: 100 - protein, 15 aa, Homo sapiens
FASPGDDRDGRAEGF

SEQ ID NO: 101 - protein, 15 aa, Homo sapiens
EPGDTALYLCASSQS

SEQ ID NO: 102 - protein, 15 aa, Homo sapiens
EPGDTALHLCASSQS

SEQ ID NO: 103 - protein, 15 aa, Homo sapiens
VNTTTSPVNTTTSPV

SEQ ID NO: 104 - protein, 15 aa, Homo sapiens
VNTTTSPANTTTSPV

SEQ ID NO: 105 - protein, 15 aa, Homo sapiens
GRKFAAWAPPSFSQT

SEQ ID NO: 106 - protein, 15 aa, Homo sapiens
GRKFAAWGPPSFSQT

SEQ ID NO: 107 - protein, 15 aa, Homo sapiens
EVPMCSDPEPRQEVP

SEQ ID NO: 108 - protein, 15 aa, Homo sapiens
EVPMCSDTEPRQEVP

SEQ ID NO: 109 - protein, 15 aa, Homo sapiens
KLSVAPSEVLEEDQV

SEQ ID NO: 110 - protein, 15 aa, Homo sapiens
KLSVAPSVVLEEDQV

SEQ ID NO: 111 - protein, 15 aa, Homo sapiens
DILEQARAAVDTYCR

SEQ ID NO: 112 - protein, 15 aa, Homo sapiens
DILEQARGAVDTYCR

SEQ ID NO: 113 - protein, 15 aa, Homo sapiens
TFNCHHARPWHNQFV

SEQ ID NO: 114 - protein, 15 aa, Homo sapiens
TFNCHHAQPWHNQFV

SEQ ID NO: 115 - protein, 15 aa, Homo sapiens
CVSMLGVPVDPDTLH

SEQ ID NO: 116 - protein, 15 aa, Homo sapiens
CVSMLGVLVDPDTLH

SEQ ID NO: 117 - protein, 15 aa, Homo sapiens
GYGEMGSGYREDLGA

SEQ ID NO: 118 - protein, 15 aa, Homo sapiens
GYGEMGSVYREDLGA

SEQ ID NO: 119 - protein, 15 aa, Homo sapiens
LLDRGSFRNDGLKAS

SEQ ID NO: 120 - protein, 15 aa, Homo sapiens
LLDRGSFWNDGLKAS

SEQ ID NO: 121 - protein, 15 aa, Homo sapiens
SQLMLTRKAEAALRK

SEQ ID NO: 122 - protein, 15 aa, Homo sapiens
SQLMLTRKGNASCLE

SEQ ID NO: 123 - protein, 15 aa, Homo sapiens
ALKIKGIHPYHSLSY

SEQ ID NO: 124 - protein, 15 aa, Homo sapiens
ALKIKGIRPYHSLSY

SEQ ID NO: 125 - protein, 15 aa, Homo sapiens
HNNIVYNEYISHREH

SEQ ID NO: 126 - protein, 15 aa, Homo sapiens
HNNIVYNKYISHREH

SEQ ID NO: 127 - protein, 15 aa, Homo sapiens
ARVILGVRWYVETTS

SEQ ID NO: 128 - protein, 15 aa, Homo sapiens
ARVILGVCWYVETTS

SEQ ID NO: 129 - protein, 15 aa, Homo sapiens
CQGDSGGPLVCGDHL

SEQ ID NO: 130 - protein, 14 aa, Homo sapiens
CQGDSGGLLVCGDH

SEQ ID NO: 131 - protein, 15 aa, Homo sapiens
PVCSGASTSCCQQSS

SEQ ID NO: 132 - protein, 15 aa, Homo sapiens
PVCSGASSSCCQQSS

SEQ ID NO: 133 - protein, 15 aa, Homo sapiens
VPVAQVTTTSTTDAD

SEQ ID NO: 134 - protein, 15 aa, Homo sapiens
VPVAQVTMTSTTDAD

SEQ ID NO: 135 - protein, 15 aa, Homo sapiens
PRCCISSCCRPSCCV

SEQ ID NO: 136 - protein, 15 aa, Homo sapiens
PRCCISSFCRPSCCV

SEQ ID NO: 137 - protein, 15 aa, Homo sapiens
CRPQCCQSVCCQPTC

SEQ ID NO: 138 - protein, 15 aa, Homo sapiens
CRPQCCQTVCCQPTC

SEQ ID NO: 139 - protein, 15 aa, Homo sapiens
TCCRTTCYRPSCCVS

SEQ ID NO: 140 - protein, 15 aa, Homo sapiens
TCCRTTCFRPSCCVS

SEQ ID NO: 141 - protein, 15 aa, Homo sapiens
PGESLRPRGERRLPQ

SEQ ID NO: 142 - protein, 15 aa, Homo sapiens
PGESLRPLGERRLPQ

SEQ ID NO: 143 - protein, 15 aa, Homo sapiens
PGSGPQNRLGRYLEV

SEQ ID NO: 144 - protein, 15 aa, Homo sapiens
PGSGPQNGLGRYLEV

SEQ ID NO: 145 - protein, 15 aa, Homo sapiens
ETGPEAERLEQLESG

SEQ ID NO: 146 - protein, 15 aa, Homo sapiens
ETGPEAEWLEQLESG

SEQ ID NO: 147 - protein, 15 aa, Homo sapiens
QKEKSLEFTKELPGY

SEQ ID NO: 148 - protein, 15 aa, Homo sapiens
QKEKSLELTKELPGY

SEQ ID NO: 149 - protein, 15 aa, Homo sapiens
PFSPSHPAPPSDPSH

SEQ ID NO: 150 - protein, 15 aa, Homo sapiens
PFSPSHPGPPSDPSH

SEQ ID NO: 151 - protein, 15 aa, Homo sapiens
DEMDCPLSPTPPLCS

SEQ ID NO: 152 - protein, 15 aa, Homo sapiens
DEMDCPLRPTPPLCS

SEQ ID NO: 153 - protein, 15 aa, Homo sapiens
VKDQGPMVSAPVKDQ

SEQ ID NO: 154 - protein, 15 aa, Homo sapiens
VKDQGPMFSAPVKDQ

SEQ ID NO: 155 - protein, 15 aa, Homo sapiens
KDQGPIVPAPVKGEG

SEQ ID NO: 156 - protein, 15 aa, Homo sapiens
KDQGPIVTAPVKGEG

SEQ ID NO: 157 - protein, 15 aa, Homo sapiens
TTTASTEGSETTTAS

SEQ ID NO: 158 - protein, 15 aa, Homo sapiens
TTTASTECSETTTAS

SEQ ID NO: 159 - protein, 15 aa, Homo sapiens
LRPQLAENKQQFRNL

SEQ ID NO: 160 - protein, 15 aa, Homo sapiens
LRPQLAEKKQQFRNL

SEQ ID NO: 161 - protein, 15 aa, Homo sapiens
EKKQQFRNLKEKCFL

SEQ ID NO: 162 - protein, 15 aa, Homo sapiens
EKKQQFRSLKEKCFL

SEQ ID NO: 163 - protein, 15 aa, Homo sapiens
AFMYAKKEEWKKAEE

SEQ ID NO: 164 - protein, 15 aa, Homo sapiens
AFMYAKKGEWKKAEE

SEQ ID NO: 165 - protein, 11 aa, Homo sapiens
KLKKKQVNVFA

SEQ ID NO: 166 - protein, 11 aa, Homo sapiens
KLKKKQVKVFA

SEQ ID NO: 167 - protein, 15 aa, Homo sapiens
GRLILWEAPPLGAGG

SEQ ID NO: 168 - protein, 15 aa, Homo sapiens
GRLILWEGPPLGAGG

SEQ ID NO: 169 - protein, 15 aa, Homo sapiens
NQLKERSFAQLISKD

SEQ ID NO: 170 - protein, 15 aa, Homo sapiens
NQLKERSIAQLISKD

SEQ ID NO: 171 - protein, 15 aa, Homo sapiens
ILLIHCDAHLHTPMY

SEQ ID NO: 172 - protein, 15 aa, Homo sapiens
ILLIHCDTHLHTPMY

SEQ ID NO: 173 - protein, 15 aa, Homo sapiens
LLIHCDAHLHTPMYF

SEQ ID NO: 174 - protein, 15 aa, Homo sapiens
LLIHCDAYLHTPMYF

SEQ ID NO: 175 - protein, 15 aa, Homo sapiens
AVVFQDSVVFRVAPW

SEQ ID NO: 176 - protein, 15 aa, Homo sapiens
AVVFQDSMVFRVAPW

SEQ ID NO: 177 - protein, 15 aa, Homo sapiens
EHSQETESLREALLS

SEQ ID NO: 178 - protein, 15 aa, Homo sapiens
EHSQETEILREALLS

SEQ ID NO: 179 - protein, 15 aa, Homo sapiens
PYGCLPTGDRTGLIE

SEQ ID NO: 180 - protein, 15 aa, Homo sapiens
PYGCLPTRDRTGLIE

SEQ ID NO: 181 - protein, 15 aa, Homo sapiens
GLPTDTIRKEFRTRM

SEQ ID NO: 182 - protein, 15 aa, Homo sapiens
GLPTDTICKEFRTRM

SEQ ID NO: 183 - protein, 15 aa, Homo sapiens
TGAMNVAKGTIQTGV

SEQ ID NO: 184 - protein, 15 aa, Homo sapiens
TGAMNVAIGTIQTGV

SEQ ID NO: 185 - protein, 15 aa, Homo sapiens
TYSPTSPVYTPTSPK

SEQ ID NO: 186 - protein, 15 aa, Homo sapiens
TYSPTSPDYTPTSPK

SEQ ID NO: 187 - protein, 15 aa, Homo sapiens
CRGSGKSNVGTSGDH

SEQ ID NO: 188 - protein, 15 aa, Homo sapiens
CRGSGKSKVGTSGDH

SEQ ID NO: 189 - protein, 15 aa, Homo sapiens
SKMGKWCRHCFAWCR

SEQ ID NO: 190 - protein, 15 aa, Homo sapiens
SKMGKWCSHCFAWCR

SEQ ID NO: 191 - protein, 15 aa, Homo sapiens
SKMGKWCRHCFPCCR

SEQ ID NO: 192 - protein, 15 aa, Homo sapiens
SKMGKWCSHCFPCCR

SEQ ID NO: 193 - protein, 15 aa, Homo sapiens
GEPIPQPARLRYVTS

SEQ ID NO: 194 - protein, 15 aa, Homo sapiens
GEPIPQPVRLRYVTS

SEQ ID NO: 195 - protein, 15 aa, Homo sapiens
VQLRGRAQGGGALRA

SEQ ID NO: 196 - protein, 15 aa, Homo sapiens
VQLRGRALGGGALRA

SEQ ID NO: 197 - protein, 15 aa, Homo sapiens
NLVHGPPAPPQVGAD

SEQ ID NO: 198 - protein, 15 aa, Homo sapiens
NLVHGPPGPPQVGAD

SEQ ID NO: 199 - protein, 15 aa, Homo sapiens
GGGPDGPLYKVSVTA

SEQ ID NO: 200 - protein, 15 aa, Homo sapiens
GGGPDGPRYKVSVTA

SEQ ID NO: 201 - protein, 15 aa, Homo sapiens
GGHDSSSWSHRYGGG

SEQ ID NO: 202 - protein, 15 aa, Homo sapiens
GGHDSSSLSHRYGGG

SEQ ID NO: 203 - protein, 15 aa, Homo sapiens
VRRCLPLCALTLEAA

SEQ ID NO: 204 - protein, 15 aa, Homo sapiens
VRRCLPLWALTLEAA

SEQ ID NO: 205 - protein, 15 aa, Homo sapiens
PSRHRYGARQPRARL

SEQ ID NO: 206 - protein, 15 aa, Homo sapiens
PSRHRYGTRQPRARL

SEQ ID NO: 207 - protein, 15 aa, Homo sapiens
ETKTKDEMAAAEEKV

SEQ ID NO: 208 - protein, 15 aa, Homo sapiens
ETKTKDETAAAEEKV

SEQ ID NO: 209 - protein, 15 aa, Homo sapiens
QEVEGETQKTEGDAQ

SEQ ID NO: 210 - protein, 15 aa, Homo sapiens
QEVEGETHKTEGDAQ

SEQ ID NO: 211 - protein, 15 aa, Homo sapiens
KSEGEEAQEVEGETQ

SEQ ID NO: 212 - protein, 15 aa, Homo sapiens
KSEGEEAHEVEGETQ

SEQ ID NO: 213 - protein, 15 aa, Homo sapiens
AGRFGQGAHHAAGQA

SEQ ID NO: 214 - protein, 15 aa, Homo sapiens
AGRFGQGDHHAAGQA

SEQ ID NO: 215 - protein, 15 aa, Homo sapiens
QLLEGLGFTLTVVPE

SEQ ID NO: 216 - protein, 15 aa, Homo sapiens
QLLEGLGCTLTVVPE

SEQ ID NO: 217 - protein, 15 aa, Homo sapiens
TRLFPNEFANFYNAV

SEQ ID NO: 218 - protein, 15 aa, Homo sapiens
TRLFPNELANFYNAV

SEQ ID NO: 219 - protein, 15 aa, Homo sapiens
QQEIDQKRLEFEKQK

SEQ ID NO: 220 - protein, 15 aa, Homo sapiens
QQEIDQKKIRINAKT

SEQ ID NO: 221 - protein, 15 aa, Homo sapiens
KELRALRKMVSNMSG

SEQ ID NO: 222 - protein, 15 aa, Homo sapiens
KELRALREMVSNMSG

SEQ ID NO: 223 - protein, 15 aa, Homo sapiens
AGQNPASHPPPDDAE

SEQ ID NO: 224 - protein, 15 aa, Homo sapiens
AGQNPASDPPPDDAE

SEQ ID NO: 225 - protein, 15 aa, Homo sapiens
PTKCEVERFTATSFG

SEQ ID NO: 226 - protein, 15 aa, Homo sapiens
PTKCEVEQFTATSFG

SEQ ID NO: 227 - protein, 15 aa, Homo sapiens
IHSSWDCGLFTNYSA

SEQ ID NO: 228 - protein, 15 aa, Homo sapiens
IHSSWDCSLFTNYSA

SEQ ID NO: 229 - protein, 15 aa, Homo sapiens
IMASKGMRHFCLISE

SEQ ID NO: 230 - protein, 15 aa, Homo sapiens
IMASKGMHHFCLISE

SEQ ID NO: 231 - protein, 15 aa, Homo sapiens
LWHLQGPKDLMLKLR

SEQ ID NO: 232 - protein, 15 aa, Homo sapiens
LWHLQGPEDLMLKLR

SEQ ID NO: 233 - protein, 15 aa, Homo sapiens
GRNSFEVRVCACPGR

SEQ ID NO: 234 - protein, 15 aa, Homo sapiens
GRNSFEVLVCACPGR

SEQ ID NO: 235 - protein, 15 aa, Homo sapiens
TSCARRDDPRASSPN

SEQ ID NO: 236 - protein, 15 aa, Homo sapiens
TSCARRDYPRASSPN

SEQ ID NO: 237 - protein, 15 aa, Homo sapiens
LGLWRGEEVTLSNPK

SEQ ID NO: 238 - protein, 15 aa, Homo sapiens
LGLWRGEAVTLSNPK

SEQ ID NO: 239 - protein, 15 aa, Homo sapiens
GCLGGENRFRLRLES

SEQ ID NO: 240 - protein, 15 aa, Homo sapiens
GCLGGENCFRLRLES

SEQ ID NO: 241 - protein, 15 aa, Homo sapiens
TQLRLPGCPTPVSFG

SEQ ID NO: 242 - protein, 15 aa, Homo sapiens
TQLRLPGWPTPVSFG

SEQ ID NO: 243 - protein, 15 aa, Homo sapiens
RKFISLHRKALESDF

SEQ ID NO: 244 - protein, 15 aa, Homo sapiens
RKFISLHKKALESDF

SEQ ID NO: 245 - protein, 15 aa, Homo sapiens
SGSGSGPLPSLFLNS

SEQ ID NO: 246 - protein, 15 aa, Homo sapiens
SGSGSGPFPSLFLNS

SEQ ID NO: 247 - protein, 15 aa, Homo sapiens
GCGKVFARSENLKIH

SEQ ID NO: 248 - protein, 15 aa, Homo sapiens
GCGKVFACSENLKIH

SEQ ID NO: 249 - protein, 15 aa, Homo sapiens
STLLTEHRRIHTGEK

SEQ ID NO: 250 - protein, 15 aa, Homo sapiens
STLLTEHLRIHTGEK

SEQ ID NO: 251 - protein, 15 aa, Homo sapiens
EKPYLCPDCGRGFGQ

SEQ ID NO: 252 - protein, 15 aa, Homo sapiens
EKPYLCPECGRGFGQ

SEQ ID NO: 253 - protein, 15 aa, Homo sapiens
EECGKPFNRFSYLTV

SEQ ID NO: 254 - protein, 15 aa, Homo sapiens
EECGKPFKRFSYLTV

SEQ ID NO: 255 - protein, 15 aa, Homo sapiens
YECNECGKAFSQSSH

SEQ ID NO: 256 - protein, 15 aa, Homo sapiens
YECNECGNAFSQSSH

SEQ ID NO: 257 - protein, 15 aa, Homo sapiens
SHNSSLILHQRVHTG

SEQ ID NO: 258 - protein, 15 aa, Homo sapiens
SHNSSLIFHQRVHTG

SEQ ID NO: 259 - protein, 15 aa, Homo sapiens
VTGGRGGRQGPSPAF

SEQ ID NO: 260 - protein, 15 aa, Homo sapiens
VTGGRGGWQGPSPAF

SEQ ID NO: 261 - protein, 15 aa, Homo sapiens
CNFSTIDVVSLKTDT

SEQ ID NO: 262 - protein, 15 aa, Homo sapiens
CNFSTIDVSLKTDTE

SEQ ID NO: 263 - protein, 15 aa, Homo sapiens
SNLTKHKKIHIEKKP

SEQ ID NO: 264 - protein, 15 aa, Homo sapiens
SNLTKHKIIHIEKKP

SEQ ID NO: 265 - protein, 15 aa, Homo sapiens
ECGQAFSLSSNLMRH

SEQ ID NO: 266 - protein, 15 aa, Homo sapiens
ECGQAFSISSNLMRH

SEQ ID NO: 267 - protein, 15 aa, Homo sapiens
IHKMIHTGEKPYKCE

SEQ ID NO: 268 - protein, 15 aa, Homo sapiens
IHKMIHTVEKPYKCE

SEQ ID NO: 269 - protein, 15 aa, Homo sapiens
CNECGKAFCQSPSLI

SEQ ID NO: 270 - protein, 15 aa, Homo sapiens
CNECGKALCQSPSLI

SEQ ID NO: 271 - protein, 15 aa, Homo sapiens
ECGKAFNRSSNLTKH

SEQ ID NO: 272 - protein, 15 aa, Homo sapiens
ECGKAFNSSSNLTKH

SEQ ID NO: 273 - protein, 15 aa, Homo sapiens
LQNHIQTIHRELVPD

SEQ ID NO: 274 - protein, 15 aa, Homo sapiens
LQNHIQTFHRELVPD

SEQ ID NO: 275 - protein, 15 aa, Homo sapiens
SNDSSLTQHQRVHTG

SEQ ID NO: 276 - protein, 15 aa, Homo sapiens
SNDSSLTHHQRVHTG

SEQ ID NO: 277 - protein, 15 aa, Homo sapiens
SNLTTHKKIHTGERP

SEQ ID NO: 278 - protein, 15 aa, Homo sapiens
SNLTTHKIIHTGERP

SEQ ID NO: 279 - protein, 15 aa, Homo sapiens
NVAKPSSGPHTLLHI

SEQ ID NO: 280 - protein, 15 aa, Homo sapiens
NVAKPSSCPHTLLHI

SEQ ID NO: 281 - protein, 15 aa, Homo sapiens
STLNTHKRIHTGEEP

SEQ ID NO: 282 - protein, 15 aa, Homo sapiens
STLNTHKSIHTGEEP

SEQ ID NO: 283 - protein, 15 aa, Homo sapiens
KCDECGNVFNWPATL

SEQ ID NO: 284 - protein, 15 aa, Homo sapiens
KCDECGNDFNWPATL

SEQ ID NO: 285 - protein, 15 aa, Homo sapiens
CKECGKAFSSSSHLI

SEQ ID NO: 286 - protein, 15 aa, Homo sapiens
CKECGKALSSSSHLI

SEQ ID NO: 287 - protein, 15 aa, Homo sapiens
HQRTHTGEKPFKCDE

SEQ ID NO: 288 - protein, 15 aa, Homo sapiens
HQRTHTGDKPFKCDE

SEQ ID NO: 289 - protein, 15 aa, Homo sapiens
EECGKAFSVFSTLTK

SEQ ID NO: 290 - protein, 15 aa, Homo sapiens
EECGKAFRVFSTLTK

SEQ ID NO: 291 - protein, 15 aa, Homo sapiens
HKRIHNGEKPYKCEE

SEQ ID NO: 292 - protein, 15 aa, Homo sapiens
HKRIHNGDKPYKCEE

SEQ ID NO: 293 - protein, 15 aa, Homo sapiens
EKPYSCPDCSLRFAY

SEQ ID NO: 294 - protein, 15 aa, Homo sapiens
EKPYSCPECSLRFAY

SEQ ID NO: 295 - protein, 15 aa, Homo sapiens
KCEECDTVFSRKSHH

SEQ ID NO: 296 - protein, 15 aa, Homo sapiens
KCEECDTDFSRKSHH

SEQ ID NO: 297 - protein, 15 aa, Homo sapiens
KAFSQSSTLRKHEII

SEQ ID NO: 298 - protein, 15 aa, Homo sapiens
KAFSQSSSLRKHEII

SEQ ID NO: 299 - protein, 15 aa, Homo sapiens
DILEQARAAVDTYCR

SEQ ID NO: 300 - protein, 15 aa, Homo sapiens
DILEQARGAVDTYCR

SEQ ID NO: 301 - protein, 22 aa, Homo sapiens
MYGCDVGSDGRFLRGYRQDAYD

SEQ ID NO: 302 - protein, 22 aa, Homo sapiens
MYGCDVGSDWRFLRGYRQDAYD

SEQ ID NO: 303 - protein, 25 aa, Homo sapiens
IQIMYGCDVGPDGRFLRGYRQDAYD

SEQ ID NO: 304 - protein, 25 aa, Homo sapiens
IQIMYGCDVGPDWRFLRGYRQDAYD

SEQ ID NO: 305 - protein, 25 aa, Homo sapiens
IQIMYGCDVGSDGRFLRGYRQDAYD

SEQ ID NO: 306 - protein, 25 aa, Homo sapiens
IQIMYGCDVGSDWRFLRGYRQDAYD

SEQ ID NO: 307 - protein, 25 aa, Homo sapiens
DVGPDGRFLRGYRQDAYDGKDYIAL

SEQ ID NO: 308 - protein, 25 aa, Homo sapiens
DVGPDGRFLRGYQQDAYDGKDYIAL

SEQ ID NO: 309 - protein, 25 aa, Homo sapiens
DVGSDGRFLRGYRQDAYDGKDYIAL

SEQ ID NO: 310 - protein, 25 aa, Homo sapiens
DVGSDGRFLRGYQQDAYDGKDYIAL

SEQ ID NO: 311 - protein, 25 aa, Homo sapiens
GSDGRFLRGYRQDAYDGKDYIALNE

SEQ ID NO: 312 - protein, 25 aa, Homo sapiens
GSDGRFLRGYRQYAYDGKDYIALNE

SEQ ID NO: 313 - protein, 25 aa, Homo sapiens
GPDGRFLRGYRQDAYDGKDYIALNE

SEQ ID NO: 314 - protein, 25 aa, Homo sapiens
GPDGRFLRGYRQYAYDGKDYIALNE

SEQ ID NO: 315 - protein, 25 aa, Homo sapiens
YLEGECVEWLRRYLENGKDKLERAG

SEQ ID NO: 316 - protein, 25 aa, Homo sapiens
YLEGECVEWLRRHLENGKDKLERAG

SEQ ID NO: 317 - protein, 25 aa, Homo sapiens
YLEGECVEWLRRYLENGKDKLERAD

SEQ ID NO: 318 - protein, 25 aa, Homo sapiens
YLEGECVEWLRRHLENGKDKLERAD

SEQ ID NO: 319 - protein, 25 aa, Homo sapiens
ALTETWACSHSMRYFDTAVSRPGRG

SEQ ID NO: 320 - protein, 25 aa, Homo sapiens
ALTETWACSHSMKYFDTAVSRPGRG

SEQ ID NO: 321 - protein, 14 aa, Homo sapiens
MRYFDTAVSRPGRG

SEQ ID NO: 322 - protein, 14 aa, Homo sapiens
MKYFDTAVSRPGRG

SEQ ID NO: 323 - protein, 17 aa, Homo sapiens
MRYFDTAVSRPGRGEPR

SEQ ID NO: 324 - protein, 17 aa, Homo sapiens
MRYFVTAVSRPGRGEPR

SEQ ID NO: 325 - protein, 17 aa, Homo sapiens
MRYFDTAVSRPGRGEPR

SEQ ID NO: 326 - protein, 17 aa, Homo sapiens
MRYFYTAVSRPGRGEPR

SEQ ID NO: 327 - protein, 19 aa, Homo sapiens
MRYFDTAVSRPGRGEPRFI

SEQ ID NO: 328 - protein, 19 aa, Homo sapiens
MRYFDTSVSRPGRGEPRFI

SEQ ID NO: 329 - protein, 25 aa, Homo sapiens
ETWACSHSMRYFDTAVSRPGRGEPR

SEQ ID NO: 330 - protein, 25 aa, Homo sapiens
ETWACSHSMRYFYTAVSRPGRGEPR

SEQ ID NO: 331 - protein, 25 aa, Homo sapiens
ETWACSHSMRYFDTAVSRPGRGEPR

SEQ ID NO: 332 - protein, 25 aa, Homo sapiens
ETWACSHSMRYFVTAVSRPGRGEPR

SEQ ID NO: 333 - protein, 25 aa, Homo sapiens
WACSHSMRYFDTAVSRPGRGEPRFI

SEQ ID NO: 334 - protein, 25 aa, Homo sapiens
WACSHSMRYFDTSVSRPGRGEPRFI

SEQ ID NO: 335 - protein, 17 aa, Homo sapiens
FRNQKGHSGLQPTGNTF

SEQ ID NO: 336 - protein, 17 aa, Homo sapiens
FRNQKGHSGLQPRGNTF

SEQ ID NO: 337 - protein, 17 aa, Homo sapiens
FRNQKGHSGLQPTGFLS

SEQ ID NO: 338 - protein, 17 aa, Homo sapiens
FRNQKGHSGLQPRGFLS

SEQ ID NO: 339 - protein, 25 aa, Homo sapiens
GAIKADHVSTYAAFVQTHRPTGEFM

SEQ ID NO: 340 - protein, 25 aa, Homo sapiens
GAIKADHVSTYAVFVQTHRPTGEFM

SEQ ID NO: 341 - protein, 25 aa, Homo sapiens
GAIKADHVSTYAAFVQTHRPTGEFM

SEQ ID NO: 342 - protein, 25 aa, Homo sapiens
GAIKADHVSTYATFVQTHRPTGEFM

SEQ ID NO: 343 - protein, 15 aa, Homo sapiens
AFTLLLYCELLQWED

SEQ ID NO: 344 - protein, 15 aa, Homo sapiens
AFTMLLYCELLQWED

SEQ ID NO: 345 - protein, 16 aa, Homo sapiens
TIYSLFYSVADRDAPA

SEQ ID NO: 346 - protein, 16 aa, Homo sapiens
TIYSLFYSVADQDAPA

SEQ ID NO: 347 - protein, 12 aa, Homo sapiens
FKQDLMIEDNLL

SEQ ID NO: 348 - protein, 12 aa, Homo sapiens
FKQDLMLEDNLL

SEQ ID NO: 349 - protein, 14 aa, Homo sapiens
QDLMIEDNLLKLEV

SEQ ID NO: 350 - protein, 14 aa, Homo sapiens
QDLMLEDNLLKLEV

SEQ ID NO: 351 - protein, 12 aa, Homo sapiens
GLLRDWRTERLF

SEQ ID NO: 352 - protein, 12 aa, Homo sapiens
GLLRYWRTERLF

SEQ ID NO: 353 - protein, 13 aa, Homo sapiens
ILFSLQPGLLRDW

SEQ ID NO: 354 - protein, 13 aa, Homo sapiens
ILFSLQPGLLRYW

SEQ ID NO: 355 - protein, 12 aa, Homo sapiens
NVSFFHYPEYGY

SEQ ID NO: 356 - protein, 12 aa, Homo sapiens
NVSFFHYQEYGY

SEQ ID NO: 357 - protein, 13 aa, Homo sapiens
EFPVRQAAAIYLK

SEQ ID NO: 358 - protein, 13 aa, Homo sapiens
EFPVLQAAAIYLK

SEQ ID NO: 359 - protein, 13 aa, Homo sapiens
INFKIERGQLLAV

SEQ ID NO: 360 - protein, 12 aa, Homo sapiens
INFKIERGQLAV

SEQ ID NO: 361 - protein, 13 aa, Homo sapiens
GAIVIERPNVKWS

SEQ ID NO: 362 - protein, 13 aa, Homo sapiens
GAIVIELPNVKWS

SEQ ID NO: 363 - protein, 14 aa, Homo sapiens
LNKVTIDARHRLPL

SEQ ID NO: 364 - protein, 14 aa, Homo sapiens
LNKVTIDAIHRLPL

SEQ ID NO: 365 - protein, 14 aa, Homo sapiens
DFGFARTLAAPGDI

SEQ ID NO: 366 - protein, 14 aa, Homo sapiens
DFGFALTLAAPGDI

SEQ ID NO: 367 - protein, 14 aa, Homo sapiens
LELMNKLLSPVVPQ

SEQ ID NO: 368 - protein, 14 aa, Homo sapiens
LELINKLLSPVVPQ

SEQ ID NO: 369 - protein, 16 aa, Homo sapiens
IEELRHLWDLLLELTL

SEQ ID NO: 370 - protein, 16 aa, Homo sapiens
IEELHHLWDLLLELTL

SEQ ID NO: 371 - protein, 15 aa, Homo sapiens
PLSQESEVEEPLSQE

SEQ ID NO: 372 - protein, 15 aa, Homo sapiens
PLSQESEMEEPLSQE

SEQ ID NO: 373 - protein, 15 aa, Homo sapiens
GFQTLTPESSCLRED

SEQ ID NO: 374 - protein, 15 aa, Homo sapiens
GFQTLTPDSSCLRED

SEQ ID NO: 375 - protein, 15 aa, Homo sapiens
EPFTTLHIQLQSGRF

SEQ ID NO: 376 - protein, 15 aa, Homo sapiens
EPFTTLHTQLQSGRF

SEQ ID NO: 377 - protein, 15 aa, Homo sapiens
DDLSRQDDNDPPKEY

SEQ ID NO: 378 - protein, 15 aa, Homo sapiens
DDLSRQDGNDPPKEY

SEQ ID NO: 379 - protein, 15 aa, Homo sapiens
FIVEQTVHAEEGIPM

SEQ ID NO: 380 - protein, 14 aa, Homo sapiens
IVEQTVQAEEGIPM

SEQ ID NO: 381 - protein, 15 aa, Homo sapiens
LAVYLPMPEDDNNSL

SEQ ID NO: 382 - protein, 15 aa, Homo sapiens
LAVYLPMSEDDNNSL

(19) United States
(12) Patent Application Publication
(10) Pub. No.: US 2021/0214686 A1
(43) Pub. Date: Jul. 15, 2021

(54) PRODUCTION AND SELECTION OF TUMOR UBER REACTIVE IMMUNE CELLS (TURICS)

(71) Applicant: POLYBIOCEPT GMBH, Heidelberg (DE)

(72) Inventors: Ernest DODOO, Heidelberg (DE); Markus MAEURER, Heidelberg (DE)

(73) Assignee: POLYBIOCEPT GMBH, Heidelberg (DE)

(21) Appl. No.: 17/264,397

(22) PCT Filed: Jul. 31, 2019
(86) PCT No.: PCT/EP2019/070696

§ 371 Date: Jan. 29, 2021

(30) Foreign Application Priority Data
Jul. 31, 2018 (EP) 18186726.8
Sep. 20, 2018 (EP) 18195826.5

(51) Int. Cl. C12N 5/0783; A61K 35/17; A61K 39/00

(52) U.S. Cl. CPC C12N 5/0636; A61K 35/17; A61K 39/0011; C12N 2502/30; C12N 2501/2315; C12N 2501/2321; A61K 2039/5158; C12N 2501/2302

(57) ABSTRACT

The present invention relates to a method for producing a T-cell product containing tumor uber reactive immune cells (TURICs) and a composition containing at least one T-cell product with TURICs for use in treatment of a cancer patient.

### FIELD OF THE INVENTION

The present invention relates to a method for producing a T-cell product containing tumor uber reactive immune cells (TURICs) and a composition containing at least one T-cell product with TURICs for use in treatment of a cancer patient.

### BACKGROUND OF THE INVENTION

The World Health Organization (WHO) reported that pancreatic cancer claimed more than 330,000 lives in 2012, with 68% of deaths occurring in countries with a high to very high human development index (HDI) (WHO, 2014). Since most patients present with metastatic disease at diagnosis, the 5-year survival rate is a meagre 5%. These statistics commensurate with limited treatment options for patients with pancreatic cancer, which include classical surgery (only 10-20% of patients qualify for this option) or chemotherapy.

A relatively new approach called adoptive cell therapy (ACT) is a progressively expanding discipline within modern oncology. This type of cell therapy relies on the transfusion of the body's own lymphocytes, thereby stimulating the patient's immune system with the intent of promoting an antigen specific anti-tumor effect. Durable clinical responses in patients with advanced cancers have been achieved using T-cells directed against tumors (tumor reactive T-cells). These approaches usually rely on the harvesting of T-cells from peripheral blood, e.g. peripheral blood mononuclear cells (PBMCs), or tumor infiltrating lymphocytes (TILs) from tumor lesions.

Since the first results of immune-based treatment of metastatic melanoma, using interleukin (IL) 2-conditioned autologous lymphocytic cells from patients' blood in the 1980s (Lotze et al, 1986; Rosenberg et al, 1985; Rosenberg et al, 1988; Topalian & Rosenberg, 1987) ACT has been further developed and has been applied to various types of cancer. Previously, it has been reported that T-cells can be reliably and successfully isolated from pancreatic cancer lesions and expanded in vitro using a cocktail of IL-2, IL-15 and IL-21 (Meng et al, 2016). Several other T-cell-based approaches to treat pancreatic cancer are currently pursued. The NCI is currently carrying out a clinical study of IL-2-stimulated TIL-infusion in patients with metastatic pancreatic cancer (ClinicalTrials.gov identifier: NCT01174121). Another study is evaluating the safety and efficacy of EGFR-directed bispecific antibody-expressing T-cells (BATs) in patients with locally advanced or metastatic pancreatic cancer who have already undergone 1-2 rounds of chemotherapy (ClinicalTrials.gov identifier: NCT03269526), although the T-cells themselves will be harvested from blood. Specialized T-cell-based therapies targeting private mutations in patients with metastatic cancers have resulted in remarkable clinical responses (Tran et al, 2015; Tran et al, 2016; Tran et al, 2014). In line with this, future clinical studies are likely to benefit translational data linking anti-tumor T-cells in pancreatic cancer to recognition of specific private mutations to improve survival as it was recently shown for a patient with metastatic breast cancer (Zacharakis et al, 2018). The activation of T-cell populations with T-cell receptors (TCRs), which specifically recognize mutated host molecules, i.e. neoepitopes, represent a clinically significant step towards refining T-cell-based immunotherapies and cancer vaccines.

Thus, it is an object of the present invention to improve and further develop personal ACTs for treatment of tumor diseases.

### SUMMARY OF THE INVENTION

The inventors identified the concept of TURICs having improved tumor reactive properties. Populations of unstimulated T-cells that reside in a precursor T-cell pool exist in body samples, which are used as common T-cell sources. These unstimulated T-cells are so low in frequency that they have not been able to proliferate in large numbers and do not exhibit classical anti-tumor activity, e.g. cytokine production, in detectable amounts after stimulation of the freshly isolated cells with tumor specific peptides or alternatively, with synthetic peptides representing the tumor mutations. The inventors have further identified that these unstimulated precursor T-cells can readily cultured and selected by the method of the invention. As shown in the Examples, when co-cultured in the presence of tumor specific peptides or autologous tumor cells, highly focused immune cells (TURICs) are generated that exhibit strong anti-tumor activity directed to these particular type of tumor cells or tumor specific peptides. The so produced TURICs exhibit a highly specific tumor reactivity resulting in anti-tumor responses above the average of T-cells, which can be isolated and proliferated with known methods.

Thus, in a first aspect, the present invention provides a method for producing a T-cell product containing tumor uber reactive immune cells (TURICs) comprising the steps of
a) providing a body sample containing T-cells of a patient;
b) optionally isolating the T-cells from the body sample;
c) stimulating the T-cells in vitro in the presence of a cytokine cocktail of the cytokines interleukin 2 (IL-2), interleukin 15 (IL-15) and interleukin 21 (IL-21) and a stimulating peptide or a group of stimulating peptides;
d) determining a reactivity factor in the T-cell sample, wherein said reactivity factor is indicative for the presence of T-cells targeting the stimulating peptide or at least one peptide of the group of stimulating peptides;
e) in case the reactivity factor is positive, identifying the T-cell sample as a tumor reactive T-cell sample; otherwise identifying the T-cell sample as a non-reactive T-cell sample;
f) culturing the non-reactive sample in vitro in the presence of the cytokine cocktail of IL-2, IL-15 and IL-21 and either one of autologous tumor cells or the stimulating peptide or the group of stimulating peptides to form a T-cell product;
g) optionally stimulating the T-cell product in vitro in the presence of the cytokine cocktail of IL-2, IL-15 and IL-21 and the stimulating peptide or the group of stimulating peptides;
h) determining the reactivity factor in the T-cell product; and
i) in case the reactivity factor is positive selecting the T-cell product as a T-cell product containing TURICs.

In a second aspect, the present invention also provides a composition containing at least one T-cell product with TURICs for use in treatment of a cancer patient, comprising performing the method according to the first aspect of the invention to obtain a T-cell product with TURICs and administering the T-cell product with TURICs to the patient.

### FIGURES

FIG. 1 shows the results of a flow cytometry analysis of TILs from patient PanTT26. (A) TILs consist of 60% CD8⁺ T-cells. After 3× stimulation of the TILs with the autologous tumor cell line, the CD8⁺ T-cell frequency increased to 99%. The results of a standard four-hour Chromium-51 release assay are shown in (B). TILs were co-incubated with the autologous tumor cell line at a ratio of 12:1 (TILs:tumor cells; represented as effector (E) to target (T) cell ratio). Parallel wells with the TIL:tumor cell co-culture were incubated with either anti-HLA class-I antibody or anti-HLA class-II antibody (anti-HLA-DR) to test for decreased tumor cell killing using the blocking antibody. While blockade of HLA class II antigen presentation partially reduced cytotoxicity of TILs, blockade of HLA-class I-restricted antigen presentation totally abrogated killing of the tumors by autologous TILs.

FIG. 2 shows the results of the characterization of T-cell product from patient PanTT39. (A) The T-cell product obtained from patient PanTT39 after IL-2, IL-15 and IL-21 stimulation stained for TCR Vβ9. After incubation with the autologous tumor cell line, the T-cell product was analyzed for induction of surface CD107a expression. Compared to the baseline, there was an 22% increase in cytotoxic activity against the autologous tumor cell line. (B) Results of HLA-classification. The T-cell product was co-cultured with the exemplary tumor specific K7N7A8-derived peptide GLLRYWRTERLF in the presence of the anti-HLA class I antibody (clone W6/32) or the anti-HLA class II antibody (clone L243). IFN-γ production was blocked by the HLA class II antibody, while anti-HLA I does not have an effect on the antigen presentation. (C) Dose-dependent activity of the T-cell product was measured by titrating an exemplary tumor specific peptide (and the corresponding wild type peptide). Targeted activity—based on peptide-driven IFN-γ production—was differentially modulated at lower concentrations of the mutated peptide. A high concentration of peptides i.e. 5 μg peptide/well/10⁵ cells, resulted in similar IFN-γ production to mutant and wild type peptides. Significant differences in IFN-γ production are present for peptide concentrations of 0.3 to 2.5 μg peptide/well/10⁵ cells.

FIG. 3 shows the results of a flow cytometry analysis of TILs from PanTT77. (A) TILs contain of almost 84% of CD4⁺ T-cells and 14% of CD8⁺ T-cells. CD4⁺ TILs were found to express the CXCR3 protein on their surface (98.8%). (B) Neoepitopes generated based on whole-exome sequencing data of the tumor tissue from patient PanTT77, were co-incubated with the autologous PBMCs or TILs over three days, after which IFN-γ production in the culture supernatants was measured. The PBMCs were found to respond to five mutated peptides while TILs reacted to nine mutated peptides. However, six mutated peptides elicited T-cell reactivity in PBMCs as well as in TILs.

FIG. 4 shows the results of flow cytometry analysis of TILs expanded from pancreatic cancer lesions. (A) 40 individual TIL lines were established and exhibited a diverse composition. Some TILs were predominantly CD3⁺CD4⁺, other CD3⁺CD8⁺; each dot represents a TIL line from an individual patient. (B)+(C) CD3⁺CD4⁺ and CD3⁺CD8⁺ TILs were gated, based on CD45RA and CCR7 expression to define the differentiation and maturation status. Most TILs resided in the central memory T-cell subset defined by CCR7⁺CD45RA⁻ expression.

FIG. 5 shows the results of flow cytometry analysis of TILs from patient PanTT39. TILs were almost 100% (99.1%) CD4⁺ T-cells, almost all of them expressing the CXCR3 protein on the surface (99.7%), which is crucial for tissue invasion/penetration.

FIG. 6 shows PBMC IFN-γ responses to peptide pools which were identified to trigger cytokine production either in PBMCs (peptide pool A; A), TILs (peptide pool B; B), or both, PBMCs and TILs (peptide pool C; C) in an initial stimulation step. The PBMCs were co-cultivated with the respective peptide pool in the presence or absence of OKT3. After 7 days of co-culture (Day 14) in the absence of OKT3, IFN-γ responses were measured. An additional measurement of IFN-γ responses was performed after another stimulation with the respective peptide pool for 3 days on day 21 of the culture in the presence or absence of OKT3. Culture A refers to PBMCs cultured with peptide pool A prior to stimulation; Culture B refers to PBMCs cultures with peptide pool B prior to stimulation; Culture C refers to PBMCs cultured with peptide pool C prior to stimulation. Peptide legend: 1: ZNF343; 2: ANKS1B; 3: PES1; 4: ZNF716; 5: ATM; 6: VCX3A; 7: PPP1R15B; 8: NBEAL1; 9: ANKS1B; 10: C2orf62; 11: CACNA15; 12: PRRT1; 13: ULBP3; 14: TMPRSS13; 15: ABCC9; 16: SFMBT2

### DETAILED DESCRIPTION OF THE INVENTION

### Definitions

The term “tumor disease” according to the invention refers to a type of abnormal and excessive growth of tissue. The term as used herein includes primary tumors and secondary tumors as well as metastasis.

A “primary tumor” according to the present application is a tumor growing at the anatomical site where tumor progression began and proceeded to yield a cancerous mass.

A “metastasis” according to the invention refers to tumors that develop at their primary site but then metastasize or spread to other parts of the body. These further tumors are also called “secondary tumors”.

A “peptide” as used herein may be composed of any number of amino acids of any type, preferably naturally occurring amino acids, which, preferably, are linked by peptide bonds. In particular, a peptide comprises at least 3 amino acids, preferably at least 5, at least 7, at least 9 amino acids. Furthermore, there is no upper limit for the length of a peptide. However, preferably, a peptide according to the invention does not exceed a length of 100 amino acids, more preferably, it does not exceed a length of 75 amino acids; even more preferably, it is not longer than 50 amino acids.

Thus, the term “peptide” includes “oligopeptides”, which usually refer to peptides with a length of 2 to 10 amino acids, and “polypeptides” which usually refer to peptides with a length of more than 10 amino acids.

A “tumor specific peptide” as used herein refers to a peptide, which is expressed only by tumor cells and thus are found in and/or on the tumor cells, but not in and/or on cells of healthy tissue. When a tumor specific peptide is only present on the surface of the tumor cell, it is also referred to as “tumor specific antigen”. Tumor specific peptides according to the invention contain an amino acid sequence with or without a mutation as found in tumor cells but not in cell of healthy tissue.

Accordingly, a tumor specific peptide as used herein is referred to as mutated or non-mutated tumor specific peptide.

As used herein an “antigen” is any structural substance, which serves as a target for the receptors of an adaptive immune response, T-cell receptor, or antibody, respectively. Antigens are in particular proteins, polysaccharides, lipids, and substructures thereof such as peptides. Lipids and nucleic acids are in particular antigenic when combined with proteins or polysaccharides.

“Disease associated antigens” are antigens involved in a disease. Accordingly, clinically relevant antigens can be tumor-associated antigens (TAA).

“Tumor associated antigens” or “TAA” according to the invention are antigens that are presented by MHC I or MHC II molecules or non-classical MHC molecules on the surface of tumor cells. As used herein, TAA includes “tumor-specific antigens”.

An “epitope” according to the invention is a portion of an antigen that is capable of stimulating an immune response. An epitope is the part of the antigen that binds to a specific antigen receptor, e.g. on the surface of an immune cell. It is possible for two or more different antigens to have an epitope in common. In these cases, the respective immune receptors are able to react with all antigens carrying the same epitope. Such antigens are known as cross-reacting antigens. “Neoepitopes”, as used herein, are newly identified epitopes, in particular epitopes of tumor associated proteins. Since each tumor carries individual mutations, a neoepitopes may only be present in one patient (individual Thutanome), giving rise to a highly personalized, antigen signature.

The terms “stimulation” or “stimulating” as used herein refer to the in vitro activation of clinically relevant lymphocytes, e.g. T-cells, by one or more stimulating agents.

Such stimulating agent may be, for example, stimulating peptides. Activation of clinically relevant lymphocytes means the onset of anti-tumor responses of these cells.

A “stimulating peptide” as used herein relates to a peptide, which is used for stimulation of T-cells. A stimulating peptide may be, for example, a tumor specific peptide, epitopes of known TAAs or neoepitopes.

“Expansion” or “clonal expansion” as used herein means production of daughter cells all arising originally from a single cell. In a clonal expansion of T-cells, all progeny share the same antigen specificity.

In agreement with the general understanding in the art “T-cell” or “T-lymphocyte”, is a type of lymphocyte (a subtype of white blood cell) that plays a central role in cell-mediated immunity. T-cells can be distinguished from other lymphocytes, such as B cells and natural killer cells, by the presence of a T-cell receptor on the cell surface. They are called T-cells because they mature in the thymus from thymocytes.

“PBMCs” as used herein refers to peripheral blood mononuclear cells, which can be obtained from peripheral blood. PBMCs mainly consist of lymphocytes, i.e. T-cells, B cells, and NK cells, and monocytes. “PBMCs” also relate to predecessor peripheral blood mononuclear cell and genetically modified cells.

“TILs” according to the invention refers to tumor infiltrating lymphocytes. These are lymphocytes, in particular T-cells predominantly found in a tumor. A lymphocyte sample derived from tumor is also referred as TIL. TILs also relate to any kind of lymphocyte that is located in, on or around a tumor or to lymphocytes that have contacted tumor tissue or tumor cells, respectively. TIL also relate to predecessor TILs and genetically modified TILs.

A “T-cell product” as used herein refers to a population of T-cells for use in immunotherapy. The “T-cell product” can be obtained by (clonal) expansion of T-cells. The T-cells can be autologous, allogeneic, or genetically modified T-cells.

The term “autologous” means that both the donor and the recipient are the same person. The term “allogenic” means that the donor and the recipient are different persons.

IL-2, IL-15 and IL-21 are members of the cytokine family each of which has a four alpha helix bundle. As used herein, “interleukin 2” or “IL-2” refers to human IL-2 and functional equivalents thereof. Functional equivalents of IL-2 include relevant substructures or fusion proteins of IL-2 that remain the functions of IL-2. Similarly, “interleukin 15” or “IL-15” refer to human IL-15 and functional equivalents thereof. Functional equivalents of IL-15 include relevant substructures or fusion proteins of IL-15 that remain the functions of IL-15. “Interleukin 21” or “IL-21” refer to human IL-21 and functional equivalents thereof. Functional equivalents of IL-21 include relevant substructures or fusion proteins of IL-21 that remain the functions of IL-21.

The term “tumor reactivity” as used herein relates to the ability of a T-cell to provide at least one of the following: containment of tumor cells, destruction of tumor cells, prevention of metastasis, stop of proliferation, stop of cellular activity, stop of progress of cells to malignant transformation, prevention of metastases and/or tumor relapse, including reprogramming of malignant cells to their non-malignant state; prevention and/or stop of negative clinical factors associated with cancer, such as malnourishment or immune suppression, stop of accumulation of mutations leading to immune escape and disease progression, including epigenetic changes, induction of long-term immune memory preventing spread of the disease or future malignant transformation affecting the target (potential tumor cells), including connective tissue and non-transformed cells that would favor tumor disease. Tumor reactive T-cells are of particular clinical/biological relevance for ACT. In contrast, T-cells, which do not provide one of the above-mentioned abilities are non-reactive.

The expressions “Tumor uber reactive immune cells” and “TURICs”, as used herein, refer to immune cells, in particular T-cells, which were specifically expanded from unstimulated precursor T-cells. TURICs recognize tumor specific peptides but not targets from healthy tissue. TURICs show stronger T-cell reactivity to a mutant peptide as compared to the corresponding non-mutated peptide. They harbor high affinity T-cell receptors and thus exhibit more exquisite tumor specificity, which also results in strongly increased anti-tumor activity compared to other T-cells from the same source or T-cell products, which can be obtained by methods known in the art.

A “reactivity factor” as used herein refers to a value obtained by assessing the tumor reactivity of T-cells either by directly measuring cytotoxic effects of the T-cells or indirectly by measuring typical T-cell responses upon treatment with tumor cells/peptides.

The transitional term “comprising”, which is synonymous with “including,” “containing,” or “characterized by,” is inclusive or open-ended and does not exclude additional, unrecited elements or method steps. The transitional phrase “consisting of” excludes any element, step, or ingredient not specified in the claim, except for impurities ordinarily associated therewith. As subject matter defined by “comprising” may contain but not necessarily contains additional, unrecited elements or method steps, any subject matter defined herein by “comprising” may be limited to “consisting of”.

Method for Producing a T-Cell Product Containing TURICs

The method according to the first aspect of the invention provides a protocol for the production of TURICs. The inventors could show that the resulting lymphocyte population after cultivation with the cytokine cocktail of IL-2, IL-15 and IL-21 in the presence of tumor specific peptides or autologous tumor cells contains a composition of lymphocytes that is advantageous for clinical application.

According to a first aspect, the invention provides a method for producing a T-cell product containing tumor uber reactive immune cells (TURICS) comprising the steps of
a) providing a body sample containing T-cells of a patient;
b) optionally isolating the T-cells from the body sample;
c) stimulating the T-cells in vitro in the presence of a cytokine cocktail of the cytokines interleukin 2 (IL-2), interleukin 15 (IL-15) and interleukin 21 (IL-21) and a stimulating peptide or a group of stimulating peptides;
d) determining a reactivity factor in the T-cell sample, wherein said reactivity factor is indicative for the presence of T-cells targeting the stimulating peptide or at least one peptide of the group of stimulating peptides;
e) in case the reactivity factor is positive, identifying the T-cell sample as a tumor reactive T-cell sample; otherwise identifying the T-cell sample as a non-reactive T-cell sample;
f) culturing the non-reactive sample in vitro in the presence of the cytokine cocktail of IL-2, IL-15 and IL-21 and either one of autologous tumor cells or the stimulating peptide or the group of stimulating peptides to form a T-cell product;
g) optionally stimulating the T-cell product in vitro in the presence of the cytokine cocktail of IL-2, IL-15 and IL-21 and the stimulating peptide or the group of stimulating peptides;
h) determining the reactivity factor in the T-cell product; and
i) in case the reactivity factor is positive selecting the T-cell product as a T-cell product containing TURICS.

Depending on the low number of unstimulated T-cells in the body sample it may be necessary to repeat the stimulation step c) for several times to increase the tumor reactivity of TURICs the initial stimulating step c) may be repeated several times, to enrich for The method according to claim 1, wherein an step c) is carried out up to three times before determining the reactivity factor in step d), preferably step c) is performed two times.

When a stimulating peptide or a group of stimulating peptides is used in the method of the invention it means that one particular type of stimulating peptide or a group of stimulating peptides of different types is applied. This does not limit the stimulating peptide to a particular number of molecules. The exact number of the applied peptide can be calculated from the given concentration. Thus, in one embodiment of the invention, the culture medium in steps c), f), and g) comprises multiple copies of a type of stimulating peptide. An increased number of copies of a stimulating peptide leads to increased expansion rate of T-cells.

In another embodiment of the invention, the culture medium in steps c), f), and g) comprises a group of stimulating peptides. The use of one or more stimulating peptides leads to a diverse set of lymphocytes, in particular T-cells reactive against the nominal clinically relevant antigen.

In the method according to the invention, either one type of stimulating peptide or a group of different types of stimulating peptides can be applied to stimulate the T-cells or for being co-cultivated with T-cells. However, when increasing the number of different types of stimulating peptides this also increases the risk of a so-called “clonal inflation”, which can lead to undesired side effects such as reduced anti-tumor activity of the resulting T-cell product. The inventors have found that in the method according to the invention up to 20 different types of stimulating peptides can be applied at the same time without significantly affecting the outcome of the method. Accordingly, in one embodiment of the present invention the group of stimulating peptides consists of up to 20 different stimulating peptides, preferably up to 10 different stimulating peptides, more preferably up to five different stimulating peptides. The group of stimulating peptides may consist of, for example, two, three, four, or five different stimulating peptides.

The T-cells/T-cell product, which are used in the method according to the first aspect, show focused recognition of several target peptides, which are identified from tumor tissue. Interestingly, the recognition of mutated tumor specific peptides triggers more pronounced anti-tumor responses than the recognition of the non-mutated peptides. Accordingly, in one embodiment of the invention, the stimulating peptides are mutated or non-mutated tumor-specific peptides, wherein the mutated tumor specific peptides contain an amino acid sequence with a mutation found in tumor cells of the patient but not in cells of healthy tissue of the patient.

Besides using tumor specific peptides or the respective tumor specific peptide epitopes as peptides alone, i.e. neoepitopes, the stimulation peptide can be represented to the T-cells in various ways. Such ways include, but are not limited to, presenting the epitopes on artificial scaffolds, as peptides with or without costimulatory molecules, with or without cytokine production of the tumor cells or other antigen-presenting cells, or autologous or allogeneic non-professional or professional cells that present the tumor epitope as a transgene or upon pulsing.

The method according to the invention uses tumor-specific peptides for stimulation of T-cells, resulting in expansion and enrichment of immune cells directed to this specific peptide. Consequently, this leads to a more precise and focused anti-tumor activity of the resulting T-cell product to tumor cells presenting said peptides. There is apparently no limitation of the type of tumor disease. Thus, the method of the invention can be used for producing a T-cell product containing TURICs directed to a huge variety of tumor diseases since peptides specific for a variety of tumors can be used, such as brain cancer, pancreas cancer, tumors derived from the neural crest, e.g. neuroblastoma, ganglioneuroma, ganglioneuroblastoma, and pheochromocytoma, epithelial, e.g. skin, lung, pancreas, colon, or breast, and mesenchymal origin, e.g. adipocytic, cartilaginous, fibrous, fibroblastic, myofibroblastic, osseous, or vascular, as well as hematopoietic tumors, e.g. blood, bone marrow, lymph, or lymphatic system.

According to an embodiment of the invention, the tumor disease is selected from brain cancer, pancreas cancer, hematopoietic tumors, tumors derived from the neural crest, and tumors of epithelial or mesenchymal origin.

Stimulation of T-cells either can be performed directly on the body samples or isolated T-cells with one or more stimulating peptides. It may be beneficial to isolate the T-cells prior to stimulation in order to provide a more selective tumor response, because of the absence of potential residual autologous stimulating agents.

Several approaches of epitope identification are currently in use, which can be used to identify new tumor associated antigens, e.g. neoepitopes. Mass spectrometry-based sequencing of peptides eluted from human leukocyte antigen (HLA) molecules derived from tumor cells aims to decipher naturally processed and presented peptides. Moreover, screening of cDNA libraries encoding TAAs has been used extensively. This peptide-based screening approach identifies mutations through whole-exome sequencing followed by in sllico analysis, based on an algorithm that predicts the peptide binding capacity of the major histocompatibility complex (MHC)-peptide complex. A further method is the tandem minigene (TMG) approach, where the patients' ‘private’ mutations are identified using whole-exome sequencing in order to subsequently construct a personalized library of gene sequences encoding mutated epitopes, e.g. neoepitopes. In the TMG setting, only a small portion of the gene around the mutation is synthesized, and its reaction with autologous T-cells is tested to identify whether the predicted neoepitopes are naturally processed and presented to the immune system based on the assumption that the surrogate antigen-presenting cells process and present the neoepitopes in a similar fashion as compared to tumor cells. The tumor specific peptides as described herein can be identified by any of the aforesaid methods or combinations thereof, e.g. as described in example 2.

Tumor antigens are mainly presented on the surface of the tumor cell via the MHC I or II complex. Since, MHC class II peptides mainly consists of 15 amino acids and most MHC class I peptides consist of 9 amino acids, rarely 8 amino acids or 10 amino acids, peptides length are chosen allowing the immune cells to decide to trim the peptide to the needed length. Accordingly, in one embodiment of the invention, the stimulating peptides have a length in the range of from 5 to 31 amino acids. The length of the stimulating peptides may be, for example, 5, 6, 7, 8, 9, 10, 11, 12, 13, 14, 15, 16, 17, 18, 19, 20, 21, 22, 23, 24, 25, 26, 27, 28, 29, 30, or 31 amino acids. In a further embodiment of the invention, the stimulating peptides have a length in the range from 7 to 25 amino acids. Preferably, the stimulating peptides have a length in the range from 9 to 21 amino acids.

In one embodiment of the invention, the tumor specific mutation is located in the middle of the peptide. In order to achieve equal trimming from both ends of the peptide, the peptide carrying the mutation in the middle preferably consists of an odd number of amino acids. Accordingly, the length of the stimulating peptides may be, for example, 5, 7, 9, 11, 13, 15, 17, 19, 21, 23, 25, 27, 29, or 31 amino acids.

In the stimulation and cultivation steps, the cells may be additionally incubated with feeder cells and/or an antibody against CD3. A co-cultivation with feeder cells and the antibody against CD3 has been described in the state of the art. It is believed that feeder cells lead to an improvement of cell growth. Feeder cells are irradiated cells that do not proliferate or proliferate only to a small extent. The feeder cells increase the number of cell contacts in the culture and additionally feed the proliferating and expanding cell culture.

The antibody against CD3 is preferably the antibody defined as OKT3. OKT3 is a murine monoclonal antibody of the immunoglobulin IgG2a isotype. The target of OKT3, CD3, is a multi-molecular complex found only on mature T-cells. An interaction between T-cells, OKT3 and monocytes causes T-cell activation in vitro.

For cytokine production and other anti-tumor reactivity assays, T-cells, are stimulated in defined cell densities. It has been reported that short-term stimulation at high cell densities, such as 10² to 10⁸ cells/μg peptide renders human T-cells from PBMCs fully reactive to soluble tumor peptides. In contrast, stimulation of T-cells in a culture with low cell density below 10² often failed to stimulate T-cells. Thus, in one embodiment of the invention, the stimulation of the T-cells and/or the T-cell product is performed, for example, on 10² to 10⁸ cells. The stimulation may be performed on 1×10² cells, 5×10² cells, 1×10³ cells, 5×10³ cells, 1×10⁴ cells, 5×10⁴ cells, 1×10⁵ cells, 5×10⁵ cells, 1×10⁶ cells, 5×10⁶ cells, 1×10⁷ cells, 5×10⁷ cells, or 1×10⁸ cells. According to another embodiment of the invention, the stimulation is performed on 10³ to 10⁶ cells, preferably on 10⁴ to 10⁵ cells.

The time of stimulation and/or cultivation of the T-cells or the T-cell product is in the range from 6 hours to 180 days. The large range of time is due to the fact that samples from different donors may behave very differently. It was shown that the lymphocytes from different body samples have very different growth rates. For example, lymphocytes derived directly from the tumor of a glioblastoma or a pancreas cancer grow very differently. From pancreas cancer derived lymphocytes are already detectable within two to five days. Lymphocytes derived from glioblastoma are only detectable after one to two weeks. Accordingly, lymphocytes from other body samples may take even longer to become detectable.

As the stimulating step(s) are performed for assessing the tumor reactivity of the T-cells, it is not required that the cells proliferate for a long period. Instead, to obtain reliable results for the analysis of tumor reactivity, a minimal duration of stimulation in the presence of the stimulating peptide(s) is required. This minimum stimulation time is dependent on the method used for determining the tumor reactivity and can vary between a few hours and several days. Similarly, the maximal time T-cells are stimulate is highly variable. It has been observed that reliable results for the determination of tumor reactivity of T-cells can be achieved from 1 hour to 10 days, depending of the determination method. Thus, according to one embodiment of the invention, the T-cells and/or the T-cell product are stimulated for 1 hour to 10 days days. For example, the cell stimulation may be carried out for 1 hour, 2 hours, 3 hours, 4 hours, 5 hours, 6 hours, 12 hours, 24 hours, 2 days, 3 days, 4 days, 5 days, 6 days, 7 days, 8 days, 9 days or 10 days. In one embodiment of the invention, the T-cells and/or the T-cell product are stimulated for 3 hours to 5 days. In another embodiment of the invention, the T-cells and/or the T-cell product are stimulated for 1 day to 3 days.

The method of the invention comprises a culturing step in culture medium either comprising autologous tumor cells or stimulating peptides, e.g. tumor specific peptides.

Due to the co-cultivation with autologous tumor cells, tumor specific T-cells are more effectively expanded. Although the culturing step can be rather short, it leads to significant improvement in the yield of expanded clinically relevant T-cells, in particular for clinically relevant T-cells expanded from peripheral blood.

It was shown that with peripheral blood cells, cultivation times about 7 days are particularly beneficial for the outcome of other cultivations. However, as mentioned above, depending on the sample an expansion of only 4 days may be enough. Given the low number of T-cells in the body sample, about 10 days or more may be necessary for co-cultivation. Thus, the culturing step is performed for 1 to 10 days. In one embodiment of the invention, the non-reactive T-cell sample is cultured with the autologous tumor cells or the stimulating peptide(s) for 1 to 10 days. The T-cell sample may be cultured, for example, for one days, 2 days, 3 days, 4 days, 5 days, 6 days, 7 days, 8 days, 9 days, or 10 days. Preferably, the non-reactive T-cell sample is cultured for 3 to 9 days, more preferably the non-reactive T-cell sample is cultured for 6 to 8 days.

In the stimulation and/or the cultivation step of the method according to the invention, the cells may undergo several rounds of expansions to expand more cells and/or to outcompete other cells with other reactivity. It has been observed that already one single round of expansion is sufficient to stimulate and/or expand the T-cells. However, up to 5 rounds of expansion have been identified to be suitable to produce T-cells for reliable stimulation/cultivation results. Thus, in one embodiment of the invention, in steps c), f) and/or g), cells undergo 1 to 5 rounds of expansion. The cells may be expanded for, for example, 1 round, 2 rounds, 3 rounds, 4 rounds, or 5 rounds.

The T-cells and the T-cell product are cultivated and/or stimulated in the presence of IL-2, IL-15, and IL-21. According to a further embodiment of the invention, the concentration of IL-2 in the liquid composition is in the range of from 10 to 6000 U/ml. The International Unit (U) is the standard measure for an amount or IL-2. It is determined by its ability to induce the proliferation of CTLL-2 cells. The concentration of IL-2 is preferably in the range from 500 to 2000 U/ml. More preferably, the concentration of IL-2 is in the range from 800 to 1100 U/ml. According to one embodiment the concentration of IL-15 is in the range of 0.1 to 100 ng/ml. preferably, the concentration of IL-15 is in the range from 2 to 50 ng/ml, more preferably in the range from 5 to 20 ng/ml. The most preferred concentration is about 10 ng/ml. In a further embodiment of the invention, the concentration of IL-21 is in the range from 0.1 ng/ml, preferably in the range from 2 to 50 ng/ml, more preferably in the range from 5 to 20 ng/ml.

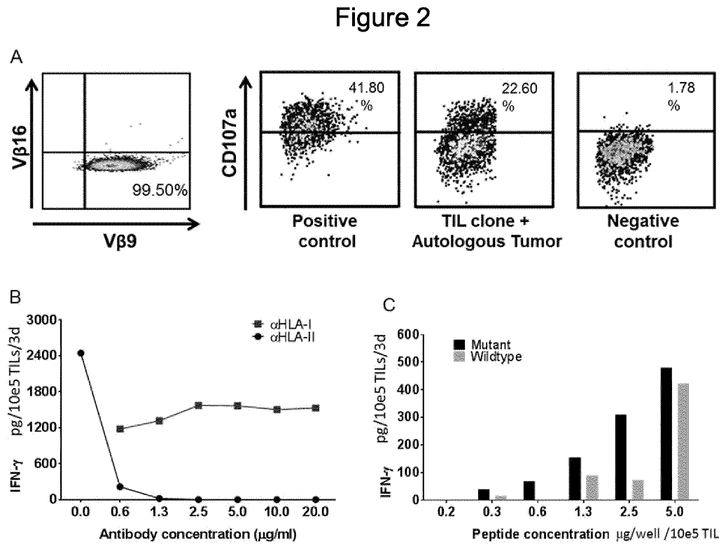

A high concentration of peptides, e.g. 5 μg peptide/well/10⁵ cells, results in detectable anti-tumor responses to mutant and wild type peptides (see FIG. 2C). However, T-cell subpopulations with TCRs that preferentially recognize private mutations can be singled out in culture when exposed to much lower peptide concentrations of from 1 pg/10⁵ cells to 1 mg/10⁵ cells.

Thus, in one embodiment, in steps c), f), and/or g) of the method, the stimulating peptide or each peptide of the group of stimulating peptides is present in a concentration of from 1 pg/10⁵ cells to 1 mg/10⁵ cells. Thus, the stimulating peptide(s) may be present, for example, in a concentration of 1 pg/10⁵ cells, 10 pg/10⁵ cells, 100 pg/10⁵ cells, 1 ng/10⁵ cells, 10 ng/10⁵ cells, 100 ng/10⁵ cells, 1 μg/10⁵ cells, 10 μg/10⁵ cells, 100 μg/10⁵ cells, or 1 mg/10⁵ cells. In another embodiment of the invention, the stimulating peptide(s) is/are in a concentration of from 1 ng/10⁵ cells to 100 μg/10⁵ cells, preferably in a concentration of 1 μg/10⁵ cells to 10 μg/10⁵ cells.

As one tumor cell is sufficient to stimulate multiple T-cells during co-cultivation, the number of autologous tumor cells in comparison to the number of T-cells is rather low. However, in order to allow for contacting all T-cells with tumor cells in the culture it also may be advantageous to use a high number of tumor cells compared to the number of T-cells. In particular, the ratio of T-cells to autologous tumor cells is in the range of from 1000:1 to 1:1000. It is found that the best results are achieved if the ratio of T-cells to autologous cancer cells is in the range of from 10:1 to 1:10. The ratio may be, for example, 10:1, 9:1, 8:1, 7:1, 6:1, 5:1, 4:1, 3:1, 1:1, 1:2, 1:3; 1:4, 1:5, 1:6, 1:7, 1:8, 1:9, or 1:10. Preferred is a ratio of from 7:1 to 3:1. Thus, in one embodiment of the invention, the non-reactive T-cell sample and the autologous tumor cells are cultured in a ratio ranging from of 1000:1 to 1:1000, preferably in a ratio ranging from of 10:1 to 1:10, more preferably in a ratio of from 7:1 to 3:1.

Repeated exposure to particular antigenic targets enrich for certain T-cell populations capable of durable anti-tumor responses (see Examples). This is based on the differential recognition of mutated peptides (as opposed to the wild type/native form), arising from important driver mutations. Accordingly, in one embodiment of the invention, step f) is carried out up to five times. Step f) may be, for example carried out once, twice, three times, four times, or five times. Preferably, step f) is performed four times, more preferably, step f) is performed three times.

Since co-culturing the T-cells to produce a T-cell product in the presence of either autologous tumor cells or stimulating peptides also triggers particular anti-tumor responses of the T-cells it is possible to directly measure anti-tumor activity of the cells after the co-culturing step of the method according to the invention. This direct measurement allows omitting an additional stimulating step before determination of the reactivity factor in step h) of the method, which reduces the overall time consumed by the method to identify and select TURICs. However, performing a stimulating step on the obtained T-cell product directly after the co-culturing step may lead to a more focused tumor reactivity and thus a more precise and consequently more reliable readout when determining the reactivity factor.

Accordingly, the method of the invention can be easily adapted to the needs of the practitioner.

Determination of parameters indicative for the presence of clinically relevant lymphocytes are known in the art and exemplified in the examples. According to one embodiment the reactivity factor is selected from T-cell proliferation, cytokine production, cytotoxicity, e.g. killing of the cells of the diseased body sample, degranulation, in particular defined by CD107a positivity, maturation and or differentiation, in particular defined by the combination of CD45RA and CCR7, expression of a T-cell activation marker in particular selected from CD25, CD56, CD69 of MHC class II molecules; an exhaustion and/or activation markers selected from Foxp3, LAG-3, TIM-3, 4-1BB, PD-1, CD127 (IL-7R), the IL-21 receptor or T-cell signaling, in particular selected from the zeta chain phosphorylation.

The testing of these parameters can be combined with flow cytometry and cell sorting. Accordingly, it is also possible to isolate the clinically relevant lymphocyte population, in particular the TURIC population from the expanded lymphocyte population. The isolated TURICs may further be cultured or directly used for immunotherapy.

One well established method to determine anti-tumor activity of immune cells is the measurement of cytokine production after stimulation with the respective stimulation peptide. Typical cytokines produced by immune cells after stimulation are e.g. IFN-γ, TNFα, IL-2, IL-17, IL-4, IL-5, GM-CSF, release of granzyme B, perforine, upregulation of activation markers, e.g. CD25, HLA-DR, CD69, 4-1BB. T-cells may produce more than one type of cytokine after stimulation simultaneously, which can be measured separately of as a whole to determine tumor reactivity of the cells. Preferred parameters indicative for the presence of clinically relevant lymphocytes are for example the production of one or more cytokines, in particular IFN-γ or TNFα production. Thus, in one embodiment of the invention, the reactivity factor is the IFN-γ concentration and the reactivity factor is positive if the concentration of IFN-γ is above a predefined IFN-γ threshold. The IFN-γ threshold used is highly variable since it depends on the experimental set-up and the culturing conditions. The threshold reflects biological relevance of the cytokine production, which—as measured ex vivo—has to be compared to a particular control experiment, e.g. medium control with or without stimulation peptides.

In one embodiment, the IFN-γ threshold is between 10 pg per 10⁵ T-cells that were stimulated with 1 μg peptide, to 150 pg/10⁵ T-cells/1 μg peptide. Accordingly, a threshold may be defined as, for example, 10 pg/10⁵ T-cells/1 μg peptide, 20 pg/10⁵ T-cells/1 μg peptide, 30 pg/10⁵ T-cells/1 μg peptide, 40 pg/10⁵ T-cells/1 μg peptide, 50 pg/10⁵ T-cells/1 μg peptide, 60 pg/10⁵ T-cells/1 μg peptide, 70 pg/10⁵ T-cells/1 μg peptide, 80 pg/10⁵ T-cells/1 μg peptide, 90 pg/10⁵ T-cells/1 μg peptide, 100 pg/10⁵ T-cells/1 μg peptide, 110 pg/10⁵ T-cells/1 μg peptide, 120 pg/10⁵ T-cells/1 μg peptide, 130 pg/10⁵ T-cells/1 μg peptide, 140 pg/10⁵ T-cells/1 μg peptide, or 150 pg/10⁵ T-cells/1 μg peptide.

In one embodiment, the reactivity factor is the CD107a positivity and the reactivity factor is positive if a T-cell is CD107a positive.

In another embodiment, the reactivity factor is T-cell proliferation, which is considered positive, when proliferation is more than two times the standard deviation of the medium control.

The direct measurement of the ability of T-cells to actively kill and destroy (autologous) tumor cells represents the most reliable assay to determine if T-cells exhibit anti-tumor activity. Thus, in another embodiment, the reactivity factor is the ability of killing tumor cells, which can be determined via a Chromium-51 release assay.

In order to test whether T-cells/T-cell products are reactive to only personal mutations of tumor specific peptides or do recognize also the non-mutated tumor specific peptide and thus may be represent a general tumor specific target, the anti-tumor activity is also tested with the non-mutated sequence peptides corresponding to the mutated tumor specific peptide, a so-called comparative peptide. Accordingly, in one embodiment of the invention, steps c) and d) are additionally carried out with a comparative peptide or a group of comparative peptides as the stimulating peptide or the group of stimulating peptides, wherein the comparative peptide contains the non-mutated sequence corresponding to the tumor specific peptide sequence.

In one embodiment of the invention, steps g) and h) are additionally carried out with a comparative peptide or a group of comparative peptides as the stimulating peptide or the group of stimulating peptides, wherein the comparative peptide contains the non-mutated amino acid sequence corresponding to the mutated tumor specific peptide sequence and wherein the T-cell product is deselected in case the reactivity factor for stimulating the T-cell product with the comparative peptide or the group of comparative peptides is equal to or higher than the reactivity factor for stimulating the T-cell product with the mutated tumor specific peptide or the group of tumor specific peptides.

Since the concentration of the stimulating peptide, e.g. the mutated tumor specific peptide or the corresponding non-mutated peptide, plays a critical role for the read out of the assays used for determining the tumor reactivity (see FIG. 2C), both peptides are applied in similar concentrations. Thus, in one embodiment of the invention, each comparative peptide is applied in a concentration similar to that of the corresponding tumor-specific peptide.

The body sample can be taken from any part of the body that contains T-cells, such as primary tumor tissue, metastasis, and peripheral blood, e.g. PBMCs. The availability of body samples for the purpose of the method of the invention can be scarce, since surgery is mostly performed for patients who present without metastasis at diagnosis. Thus, the use of PBMCs to screen for neoepitope recognition is also a viable approach for developing personalized cellular therapies. Accordingly, in one preferred embodiment of the invention, the body sample is whole blood, in particular the body sample are PBMCs.

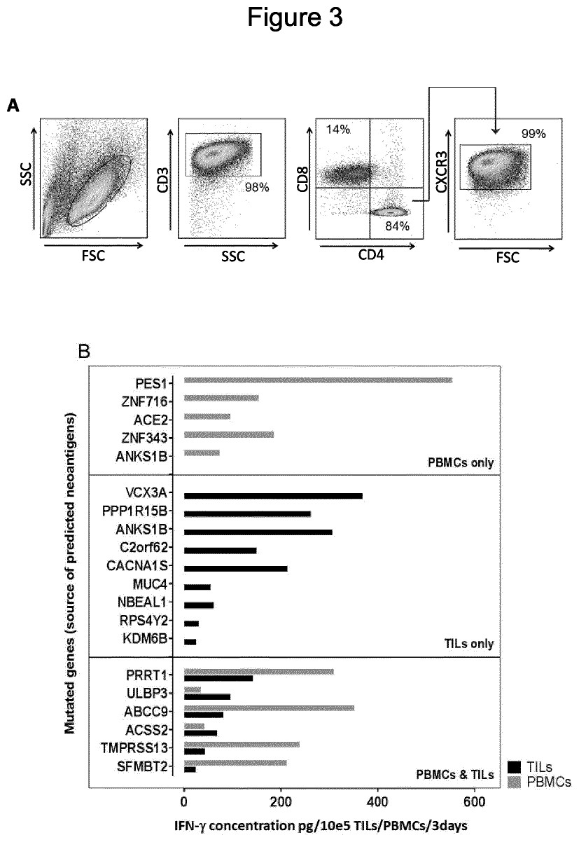

Moreover, it was found that some stimulating peptides are recognized by both, T-cells from PBMCs and T-cells from TILs, but there are also tumor specific peptides, which are either exclusively recognized by PBMC or TIL T-cells (see FIG. 3B). Interestingly, after co-culturing PBMC T-cells, which do not respond to a stimulating peptide that triggers anti-tumor responses in TIL T-cells, with autologous tumor cells, the resulting PBMC T-cell product strongly respond to the initially not recognized peptides. This response of co-cultured T-cells to peptides, to which T-cells prior to co-cultivation do not respond, shows that the presence of unstimulated T-cell populations and thus the concept of TURICs as described herein is not limited to one particular body sample. This means that in the event no elevated anti-tumor responses can be detected for T-cells of one type of body sample, the method according to the invention can be performed on another type of body sample from the same patient in order to identify and select TURICs.

Thus, in one embodiment of the invention, the method can be performed on actually an unlimited number of body samples. Preferably, the method can be performed on at least 2, at least 3, at least 4, at least 5, or at least 6 body samples. In particular, the method can be performed on 1, 2, 3, 4, 5, 6, 7, 8, 9, or 10 body samples.

One advantage of the present invention is that there is no need to provide T-cells from tumor tissue. This is in particular useful, because it enables the practitioner to obtain T-cells from non-tumor samples. These body samples can be easily obtained without performing surgery and thus preventing risks associated with such medical interventions. Accordingly, in one embodiment of the invention, the body sample is not a tumor a sample.

In a further embodiment, the body sample is selected from whole blood, serum, plasma, urine, tears, sperm, saliva, synovial fluid, umbilical cord, placenta tissue, bone marrow, exhaled air, lavage material, such as bronchoalveolar lavage, cerebrospinal fluid (CSF), primary, secondary lymphoid tissues, samples from the gut lumen, samples from peritoneal cavity, transplanted material, transplanted cells, transplanted tissue(s) or organ(s).

Methods for obtaining T-cells are known in the art. For example, T-cells can be isolated during surgical interventions such as biopsies. T-cells can also be isolated by aspiration of single cells from tissues and/or organs.

T-cells can be stimulated in the presence of IL-2, IL-15, and IL-21 directly after isolation from the body sample. Moreover, it is also possible to store the freshly isolated T-cells or the obtained T-cell product until use, e.g. by freezing.

Composition for Treatment

The inventors observed that the T-cell product obtained by the method according to the first aspect exhibit elevated therapeutic effects. Thus, in a second aspect, the invention provides a composition containing at least one T-cell product with TURICs for use in treatment of a cancer patient, comprising performing the method according to the first aspect to obtain a T-cell product with TURICs and administering the T-cell product with TURICs to the patient.

Since TURICs are considered immune cells with a tightly focused anti-tumor activity and, thus, are not limited to any specific type of cancer, they can be used in the treatment of literally any tumor disease. As a result, the composition according to the second aspect, can be used for the treatment of diverse tumor diseases such as brain cancer, pancreas cancer, tumors derived from the neural crest, e.g. neuroblastoma, ganglioneuroma, ganglioneuroblastoma, and pheochromocytoma, epithelial, e.g. skin, colon, or breast, and mesenchymal origin, e.g. adipocytic, cartilaginous, fibrous, fibroblastic, myofibroblastic, osseous, or vascular, as well as hematopoietic tumors, e.g. blood, bone marrow, lymph, or lymphatic system.

According to an embodiment of the invention, the tumor disease is selected from brain cancer, pancreas cancer, hematopoietic tumors, tumors derived from the neural crest, and tumors of epithelial or mesenchymal origin.

The T-cell product has a low percentage of regulatory T-cells. Regulatory T-cells are known to suppress the therapeutic function of the population of lymphocytes. According to one embodiment of the second aspect the T-cell product the percentage of T_{reg} based on the total number of T-cells is below 5%, preferably below 3%.

An effective amount of the T-cell product containing TURICs, or compositions thereof, can be administered to the patient in need of the treatment via a suitable route, such as, for example, intravenous administration. The cells may be introduced by injection, catheter, or the like. If desired, additional drugs (e.g., cytokines) may also be co-introduced or introduced sequentially. Any of the cells or compositions thereof may be administered to a subject in an effective amount. As used herein, an effective amount refers to the amount of the respective agent (e.g., the cells or compositions thereof) that upon administration confers a desirable therapeutic effect on the subject. Determination of whether an amount of the cells or compositions described herein achieved the desired therapeutic effect would be evident to one of skill in the art.

The composition containing the T-cell product can be delivered by using administration routes known in the art. Suitable administrations routes are, for example, intravenous administration, subcutaneous administration, intra-arterial administration, intradermal administration, intrathecal administration.

Preferably, the composition containing the T-cell product is administered via the intravenous route, intra-arterial route, intrathecal route, or intraperitoneal route, or directly into the tissue, directly in the bone marrow, or into the cerebrospinal fluid via a catheter.

The person skilled in the art is aware of different formulations of the composition containing the T-cell product to be administered. As such, exemplary formulations may contain polyethylene glycol (PEG) or other substances supporting and/or facilitating the administration of the composition.

Moreover, the compounds administered can be obtained by well-known methods. Such methods may be, for example, production of proteins by recombinant means. Additionally, recombinant proteins can be produced in a variety of cell types that have been adapted to the production of recombinant proteins. Those cells can be transfected with the genetic construct of the respective protein to be produced by methods known in the art, e.g. retroviral, non-retroviral vectors, or CRISP-Cas9 based methods.

According to an embodiment of the present invention, the patient is administered with a dose of TURICs of from 10⁷ to 10⁸ cells per kg body weight. The dose of TURICs may be for example 1×10⁷, 1.5×10⁷, 2×10⁷, 2.5×10⁷, 3×10⁷, 3.5×10⁷, 4×10⁷, 4.5×10⁷, 5×10⁷, 5.5×10⁷, 6×10⁷, 6.5×10⁷, 7×10⁷, 7.5×10⁷, 8×10⁷, 8.5×10⁷, 9×10⁷, 9.5×10⁷, or 1×10⁸ of cells per kilogram body weight.

The invention is further defined by the following non-limiting examples.

### EXAMPLES

### Example 1—Isolation and Generation of T-Cells and Autologous Tumor Cells

Pancreatic cancer TILs and autologous tumor cell lines were obtained from three patients with pancreatic cancer.

1.1 Generation of TILs

Individual single tumor fragments (1-2 mm³) were placed in each well of a 24-well tissue culture plate along with TIL medium, i.e. Cellgro GMP Serum-free medium (CellGenix, Freiburg, Germany), with 5% human AB serum (Innovative Research, Michigan, USA), supplemented with IL-2 1000 IU/ml, IL-15 10 ng/ml, IL-21 10 ng/ml, (Prospec, Ness-Ziona, Israel). Irradiated (55 Gry) feeder cells (allogeneic PBMCs) at the ratio of 1 (feeder cells):10 (TILs) was added on day 7. TILs were transferred into six-well plates; as they covered >70% of the 24-well surface, they were further expanded in G-Rex flasks (Wilson Wolf, Catalog Number: 800400S) using 30 ng OKT3/mL and irradiated (55 Gry) allogeneic feeder cells at the ratio of 1 (feeder cells):5 (TILs).

1.2 Generation of Autologous Tumor Cells

After surgery or biopsy, tumor tissues were cut with surgical scissors and a scalpel. Single tumor fragments (1-2 mm³) were placed in 24-well tissue culture plates with 1 ml of tumor medium, i.e. RPMI 1640 with 20% FBS (Life technologies, CA, USA), Epidermal growth factor (20 ng/ml, ImmunoTools, Friesoythe, Germany) supplemented with antibiotics (penicillin, 100 IU/mL and streptomycin, 10 mg/mL) (Life Technologies, Carlsbad, USA) and Amphotericin B (2.5 mg/L, Sigma-Aldrich, MI; USA). Tumor cell lines were then cultured and passed without EGF. Tumor cells required for the cytotoxicity experiments were obtained during passage fifteen to twenty.

1.3 PBMC Isolation

Peripheral blood mononuclear cells (PBMCs) were isolated from whole blood over a Ficoll-Hypaque gradient (GE Healthcare, Uppsala, Sweden) and washed twice in sterile PBS prior to use in experiments.

### Example 2—DNA Isolation, Whole-Genome Sequencing, Mutanome Analysis and Neoepitope Synthesis

Isolation and purification of genomic DNA, library construction, exome capture of all coding genes as well as next generation sequencing of tumor tissue and control patient samples were performed as briefly described in the following. Genomic DNA from patient samples (tumor tissue and TILs) was fragmented for constructing an Illumina DNA library (Illumina, San Diego, Calif.). Regions of DNA corresponding to exons were captured in solution using the Agilent SureSelect 50 Mb kit Version 3 as per manufacturer's instructions (Agilent, Santa Clara, Calif.). Paired-end sequencing resulting in 100 bases from each end of every fragment was performed using a HiSeq 2000 Genome Analyser (Illumina, San Diego, Calif.). Results of the sequencing data were mapped to the reference human genome sequence. Alterations within the sequencing data were determined by comparing over 50 million bases of tumor DNA from non-malignant lesions. A high fraction of the sequences obtained for each sample was found to occur within the captured coding regions. More than 43 million bases of target DNA were analyzed in the tumor and normal samples; an average of 42 to 51 reads per base was obtained for both sample types. The tags were aligned to the human genome reference sequence (hg18) using the Eland algorithm of CASAVA 1.6 software (Illumina, San Diego, Calif.). The chastity filter of the BaseCall software of Illumina was used to select sequence reads for subsequent analysis. The ELANDv2 algorithm of CASAVA 1.6 software (Illumina, San Diego, Calif.) was applied for identifying point mutations, small insertions, deletions or stop codons in the sequences obtained. Mutation polymorphisms recorded in the Single Nucleotide Polymorphism Database (dbSNP) were excluded from analysis. Potential somatic mutations were filtered out as previously described, (Jones et al, 2010) while only non-synonymous single and dinucleotide substitutions, respectively were listed in an Excel spreadsheet for downstream work.

The filter criterion for selecting candidate peptides is that the expression level of mutated genes in tumor tissue surpasses 5%. Alternatively, spliced products or mutated sequences with stop codons may result in epitopes that are shorter than the standard 15-mer peptides that are used for screening immunogenicity. The length of the resulting peptide sequences was set at 15-mer to include all possible epitopes presented by HLA class I (8-10 amino acids) as well as HLA class II (11-20 amino acids) molecules.

After identification of mutations through whole-exome sequencing followed by in sllico analysis, the 15-mer peptides were constructed by placing the mutation at the centre position of the 15-amino acid sequence (Peptide & Elephants, Berlin, Germany). The corresponding wild type epitopes were also synthesized to compare the matched mutant and wild type sequences (peptide pairs) in immunological assays.

### Example 3—Evaluation of the Immunoreactivity of T-Cells to Neoepitopes

3.1 IFN-γ Production

T-cells (1.0×10⁵ cells), e.g. from TILs or PBMCs, were cultured in 200 μl of T-cell medium with 1 μg of the individual wild type or mutated peptide in round-bottom 96-well microtiter plates. Negative controls contained assay medium alone while the positive control contained 30 ng/mL of the anti-human CD3 antibody clone OKT3 (Biolegend, San Diego, Calif.) for maximal TCR stimulation. Cells were incubated for 3 days at 37° C. with 5% CO₂, after which supernatants were harvested for IFN-γ production using a standard sandwich enzyme-linked immunosorbent assay (ELISA) kit (Mabtech, Stockholm, Sweden). Values from the negative control (medium) were subtracted from epitope (peptide)-specific responses and the data reported to reflect the IFN-γ production (in pg/3 days/1.0×10⁵ T-cells) representing the net IFN-γ production from T-cell populations. Where necessary, the mAbs w6/32 (anti-MHC class I, HLA-A, B and -C) and L243 (anti-HLA-DR) were used as blocking antibodies to assess MHC class I or—class I restriction.

3.2 CD107a Induction Assay

T-cells (2×10⁵) were co-cultured with 4×10⁴ autologous tumor cells for 5 hours at 37° C. (and 5% CO₂) in a 96-well tissue culture plate containing 200 μl assay medium/well (RPMI 1640 with 10% FBS and penicillin/streptomycin; both from Thermo Fisher Scientific, Waltham, Mass.). During the incubation period, 1.3 μg/ml of monensin (Merck KGaA, Darmstadt, Germany), and 4 μl of the anti-human CD107a-Alexa Fluor 700 antibody (Clone H4A3; BD Biosciences, Franklin Lakes, N.J.) were added. PMA was used as the positive control and assay medium alone without tumor cells was used as negative control. After 5 hours of incubation, the cells were stained with anti-human CD3-PE/Cy7 (Clone HIT3A; BioLegend, San Diego, Calif.), anti-human CD4-V450 (Clone RPA-T4) and anti-human CD8-APC/Cy7 (Clone SK1) (both from BD Biosciences, Franklin Lakes, N.J.), and analyzed by flow cytometry.

3.3 Chromium-51 Release Assay

Specific cytotoxicity was determined in a standard Chromium-51 (Cr⁵¹) release assay. Autologous or control tumor cell lines (‘target cells’, T) were labeled with 100 μCi Na₂ ⁵¹CrO₄ for 2 hours. T-cells were co-incubated with the autologous tumor cell line at a ratio of 12:1 (T-cell:tumor cells; represented as effector (E) to target (T) cell ratio). Parallel wells with the T-cell:tumor cell co-culture were incubated with either anti-HLA class-I antibody (clone W6/32) or anti-HLA class-II antibody (clone L243, anti-HLA-DR) to test for decreased tumor cell killing using the blocking antibody (interfering with the MHC Class I or MHC class II antigen presentation). Chromium-51 release was measured in the supernatant and specific cytotoxic activity was calculated by the standard method.

3.4 Repeated Stimulation with the Autologous Tumor Cell Line

For repeated stimulation with the autologous tumor cell line, T-cells were co-cultivated with the tumor cells in six-well tissue culture plates (5×10⁶ TILs: 1×10⁶ tumor cells) containing T-cell medium for seven days, after which TILs were stimulated with tumor cells two more times, thereby forming a T-cell product.

After the repeated stimulation with tumor cells, the resulting T-cell product was assessed for immunoreactivity either directly after co-cultivation with the tumor cells for 7 days or following the above stimulation in the presence of the mutated peptide.

### Example 4—Phenotyping of T-Cells in the T-Cell Product

4.1 Flow Cytometry and Analysis

All flow cytometry experiments were performed on a BD FACS Aria flow cytometer while data analysis was performed using FlowJo software version 7 (both from BD Biosciences, Franklin Lakes, N.J.).

4.2 T-Cell Phenotype

T-cells were stained with anti-CD3 Brilliant violet 570, anti-CD4 Brilliant violet 510, anti-CXCR3 FITC (all from Biolegend, San Diego, Calif.) and anti-CD8a APC-Cy7 (BD Biosciences, Franklin Lakes, N.J.). After 15 minutes, cells were washed in PBS-0.1% FBS, and analyzed by flow cytometry. Differentiation and maturation marker analysis based on CD45RA and CCR7 expressed was performed as described previously (Liu et al, 2016).

Results of Examples 1 to 4

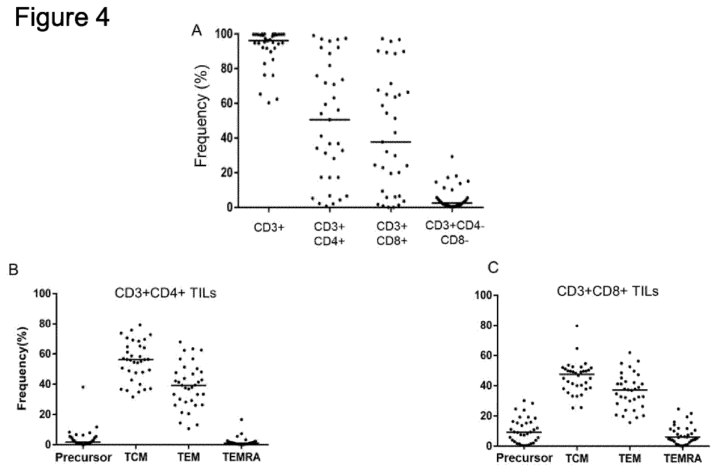

To better facilitate presentation of data relevant to the present study, the results section has been organised to reflect the findings pertinent to each patient individually. The reliable expansion of CD4⁺ and CD8⁺ TILs from pancreatic cancer tissue using IL-2, IL-15 and IL-21, particularly within the central and effector memory compartments, is shown in FIG. 4.

Patient Pan TT26

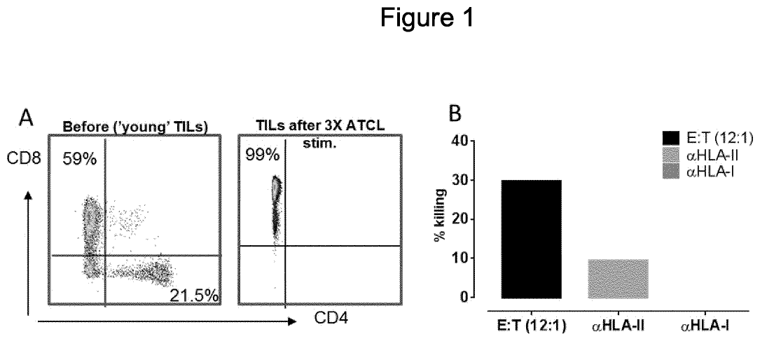

TILs and the corresponding tumor cell line was established from patient PanTT26. Flow cytometry analysis revealed that TILs before stimulation with autologous tumor cells (“young” TILs) comprised approximately 59% CD8⁺ T-cells and 22% CD4⁺ T-cells (FIG. 1A). The TILs were then stimulated with the PanTT26 tumor cell line (autologous) three times to see whether repeated exposure of the IL-2/IL-15/IL-21-conditioned TILs to the tumor would lead to enrichment of tumor epitope-reactive T-cells. The resulting TILs were enriched for CD8⁺ TILs (almost 100%) while CD4⁺ T-cells were entirely absent.

Using the W6/32 (anti-HLA-I) and L243 (anti-HLA-DR) antibodies in Cr51-release assays, it was found that TILs, prior to 3× stimulation with the autologous tumor cell line, displayed a dampened cytotoxic effect with HLA class II inhibition while the W6/32 (anti-MHC class I) antibody abrogated tumor recognition completely (FIG. 1B). This observation indicated that the cytotoxic effect of PanTT26 TILs was mainly restricted by HLA class I antigen presentation.

Whole-exome sequencing of the pancreatic tumor tissue from patient PanTT26 was performed to identify cancer-related mutations that may give rise to mutated antigens (neoantigens). The identified peptide sequences were synthesized along with the corresponding wild type sequences and tested for T-cell reactivity by measuring antigen-specific IFN-γ production. In total, 298 peptides (149 wild type and mutated, respectively) were identified and tested for both TILs before (“young TILs) and after 3× stimulation with the autologous tumor cells. It was found that more than 150 pg IFN-γ (per 10⁵ T-cells/1 microgram peptide) was produced by young TIL or tumor cell-stimulated TIL in response to subsequent exposure to wild type or mutated peptides as it is shown in Table 1.

TABLE 1
IFN-γ production by PanTT26 TILs to all predicted private mutated
Targets (and the corresponding wild type sequences) before and after
repeated stimulation with the autologous tumor cell line.
Legend: WT = wild type; Mut = mutant

| | | | | IFN-γ Pg/10⁵ TIL/1 | | | |
|---|---|---|---|---|---|---|---|
| | | | | microgram peptide | | | |
| | | | | | | Stimulated | |
| | | | | | | 3 times | |
| | | | | Young | | with tumor | |
| | Wild type | Mutated | Gene | TIL | | cell line | |
| Peptide ID | sequence | sequence | Name | WT | Mut | WT | Mut |
| PanTT26-P1 | FEGTEMWNPNRELSE | FEGTEMWYPNRELSE | ACHE | 104 | 14 | 228 | 0 |
| PanTT26-P2 | AVKRLPLVYCDYHGH | AVKRLPLIYCDYHGH | AGTPBP1 | 60 | 149 | 0 | 0 |
| PanTT26-P3 | TCSCQSSGTSSTSYS | TCSCQSSWTSSTSYS | AK302451 | 30 | 0 | 0 | 0 |
| PanTT26-P4 | PWRKFPVYVLGQFLG | PWRKFPVHVLGQFLG | AQP7 | 152 | 315 | 0 | 443 |
| PanTT26-P5 | MNAAVTFANCALGRV | MNAAVTFTNCALGRV | AQP7 | 41 | 0 | 0 | 119 |
| PanTT26-P6 | INCLSSPNEETVLSA | INCLSSPSEETVLSA | ARMC7 | 60 | 0 | 0 | 0 |
| PanTT26-P7 | STAYPAPMRRRCCLP | STAYPAPVRRRCCLP | ARMC7 | 261 | 122 | 446 | 0 |
| PanTT26-P8 | VALKPQERVEKRQTP | VALKPQECVEKRQTP | AUTS2 | 94 | 0 | 167 | 0 |
| PanTT26-P9 | PSHQPPASTLSPNPT | PSHQPPARTLSPNPT | C5orf60 | 24 | 0 | 0 | 0 |
| PanTT26-P10 | TPEPAIPPKATLWPA | TPEPAIPHKATLWPA | C6orf132 | 156 | 0 | 0 | 0 |
| PanTT26-P11 | MFTLTGCRLVEKT | MFTLTSCRLVEKT | CCDC108 | 12 | 0 | 0 | 0 |
| PanTT26-P12 | THRPGGKHGRLAGGS | THRPGGKRGRLAGGS | CCDC74B | 104 | 114 | 0 | 487 |
| PanTT26-P13 | VTVHPTSNSTATSQG | VTVHPTSKSTATSQG | CD68 | 41 | 0 | 0 | 281 |
| PanTT26-P14 | STATHSPATTSHGNA | STATHSPSTTSHGNA | CD68 | 36 | 0 | 0 | 0 |
| PanTT26-P15 | LQREYASVKEENERL | LQREYASMKEENERL | CDK5RAP2 | 77 | 0 | 0 | 0 |
| PanTT26-P16 | MEVSGCPTPAGQS | MEVSGWPTPAGQS | CEACAM18 | 75 | 0 | 98 | 0 |
| PanTT26-P17 | ARAAAAAAFEIDPRS | ARAAAAATFEIDPRS | CELSR3 | 17 | 0 | 0 | 0 |
| PanTT26-P18 | HGLSHSLRQISSQLS | HGLSHSLWQISSQLS | CEP164 | 49 | 15 | 0 | 0 |
| PanTT26-P19 | VTTKKTPPSQPPGNV | VTTKKTPSSQPPGNV | CNTN3 | 17 | 0 | 0 | 0 |
| PanTT26-P20 | SSLPGPPGPPGPPGP | SSLPGPPGPPGPRGY | COL18A1 | 4 | 1 | 0 | 169 |
| PanTT26-P21 | FSISQLQKNHDMNDE | FSISQLQTNHDMNDE | CYP7B1 | 78 | 22 | 0 | 149 |
| PanTT26-P22 | GINQTTGALYLRVDS | GINQTTGTLYLRVDS | DCHS1 | 12 | 0 | 0 | 0 |
| PanTT26-P23 | MSYDYHQNWGRDGG | MSYDYHHNWGRDGG | DHX36 | 110 | 46 | 164 | 0 |
| PanTT26-P24 | EMQEERLKLPILSEE | EMQEERLTLPILSEE | DHX37 | 70 | 0 | 41 | 0 |
| PanTT26-P25 | ENKNQELRSLISQYQ | ENKNQELHSLISQYQ | DOCK3 | 8 | 0 | 0 | 0 |
| PanTT26-P26 | SSAEPTEHGERTPLA | SSAEPTENGERTPLA | DPCR1 | 10 | 0 | 0 | 0 |
| PanTT26-P27 | VLACGLSRIWGEERG | VLACGLSQIWGEERG | EDNRB | 66 | 0 | 0 | 0 |
| PanTT26-P28 | LADGEGGGTDEGIYD | LADGEGGATDEGIYD | EFS | 20 | 190 | 0 | 0 |
| PanTT26-P29 | YVVPPPARPCPTSGP | YVVPPPAWPCPTSGP | EFS | 47 | 0 | 0 | 0 |
| PanTT26-P30 | MRETSGFTL | MRDKWLHIE | PRSS3 | 77 | 0 | 0 | 0 |
| PanTT26-P31 | ECSECGKVFLESAAL | ECSECGKDFLESAAL | ZNF264 | 81 | 0 | 0 | 0 |
| PanTT26-P32 | LTDHRAHRCPGDGDD | LTDHRAHCCPGDGDD | ZNF423 | 45 | 0 | 0 | 0 |
| PanTT26-P33 | LTDHRAHRCPGGNAK | LTDHRAHCCPGGNAK | ZNF423 | 72 | 18 | 0 | 0 |
| PanTT26-P34 | DEVSMKGRPPPTPLF | DEVSMKGGPPPTPLF | FKBP15 | 63 | 0 | 0 | 0 |
| PanTT26-P35 | AQGWSTVARFQITAT | AQGWSTVSRFQITAT | SLC25A23 | 107 | 0 | 0 | 0 |
| PanTT26-P36 | KLVVVGAGGVGKSAL | KLVVVGAVGVGKSAL | KRAS | 54 | 0 | 0 | 453 |
| PanTT26-P37 | LFGLGKDEGWGPPAR | LFGLGKDVGWGPPAR | NT5C3B | 65 | 100 | 0 | 3 |
| PanTT26-P38 | VMMHGGPPHPGMPMS | VMMHGGPAHPGMPMS | MEIS1 | 92 | 5 | 0 | 0 |
| PanTT26-P39 | MRHFCLISE | MHHFCLISE | TMEM168 | 124 | 0 | 355 | 0 |
| PanTT26-P40 | PMEKPTISTEKPTIP | PMEKPTITTEKPTIP | ZAN | 13 | 3 | 0 | 0 |
| PanTT26-P41 | YVSMMCNEQAYSLAV | YVSMMCNKQAYSLAV | NDUFS2 | 24 | 50 | 16 | 0 |
| PanTT26-P42 | LWTEGMLQMAFHILA | LWTEGMLKMAFHILA | UBR1 | 7 | 71 | 95 | 0 |
| PanTT26-P43 | KPVILGVRWYVETTS | KPVILGVCWYVETTS | KLK6 | 57 | 16 | 166 | 0 |
| PanTT26-P44 | TMLARLVSDS | TMLARLVLDS | FAM161A | 63 | 33 | 0 | 0 |
| PanTT26-P45 | SSGGGSSGGGYGGGS | SSGGGSSSGGYGGGS | KRT10 | 94 | 19 | 242 | 0 |
| PanTT26-P46 | DPSAIGLADPPIPSP | DPSAIGLVDPPIPSP | SELV | 114 | 20 | 37 | 0 |
| PanTT26-P47 | QLTAHKMIHTGEKPY | QLTAHKMNHTGEKPY | ZNF100 | 46 | 45 | 0 | 0 |
| PanTT26-P48 | AVYTPPSVSTHQMPR | AVYTPPSDSTHQMPR | PRSS3 | 22 | 17 | 0 | 0 |
| PanTT26-P49 | PGSGPQNPPGLGSGA | PGSGPQNAPGLGSGA | LILRB3 | 0 | 0 | 0 | 0 |
| PanTT26-P50 | FASPGDDGDGRAEGF | FASPGDDRDGRAEGF | MUC12 | 9 | 12 | 0 | 0 |
| PanTT26-P51 | EPGDTALYLCASSQS | EPGDTALHLCASSQS | TRBV23-1 | 6 | 37 | 0 | 0 |
| PanTT26-P52 | VNTTTSPVNTTTSPV | VNTTTSPANTTTSPV | M54A18 | 46 | 20 | 0 | 79 |
| PanTT26-P53 | GRKFAAWAPPSFSQT | GRKFAAWGPPSFSQT | PTX4 | 240 | 94 | 457 | 0 |
| PanTT26-P54 | EVPMCSDPEPRQEVP | EVPMCSDTEPRQEVP | FAM120B | 78 | 0 | 0 | 0 |
| PanTT26-P55 | KLSVAPSEVLEEDQV | KLSVAPSVVLEEDQV | GGTA1P | 19 | 2 | 0 | 0 |
| PanTT26-P56 | DILEQARAAVDTYCR | DILEQARGAVDTYCR | HLA-DRB1 | 25 | 0 | 0 | 0 |
| PanTT26-P57 | TFNCHHARPWHNQFV | TFNCHHAQPWHNQFV | HTR3D | 60 | 34 | 0 | 0 |
| PanTT26-P58 | CVSMLGVPVDPDTLH | CVSMLGVLVDPDTLH | HUWE1 | 0 | 5 | 0 | 0 |
| PanTT26-P59 | GYGEMGSGYREDLGA | GYGEMGSVYREDLGA | IGFN1 | 21 | 39 | 0 | 488 |
| PanTT26-P60 | LLDRGSFRNDGLKAS | LLDRGSFWNDGLKAS | KALRN | 42 | 87 | 0 | 354 |
| PanTT26-P61 | SQLMLTRKAEAALRK | SQLMLTRKGNASCLE | KANSL1 | 196 | 0 | 74 | 0 |
| PanTT26-P62 | ALKIKGIHPYHSLSY | ALKIKGIRPYHSLSY | KIAA1109 | 177 | 139 | 65 | 0 |
| PanTT26-P63 | HNNIVYNEYISHREH | HNNIVYNKYISHREH | KIN | 0 | 5 | 0 | 0 |
| PanTT26-P64 | ARVILGVRWYVETTS | ARVILGVCWYVETTS | KLK6 | 40 | 28 | 0 | 62 |
| PanTT26-P65 | CQGDSGGPLVCGDHL | CQGDSGGLLVCGDHL | KLK6 | 27 | 0 | 0 | 0 |
| PanTT26-P66 | PVCSGASTSCCQQSS | PVCSGASSSCCQQSS | KRTAP10-5 | 0 | 7 | 0 | 0 |
| PanTT26-P67 | VPVAQVTTTSTTDAD | VPVAQVTMTSTTDAD | KRTAP11-1 | 25 | 44 | 0 | 103 |
| PanTT26-P68 | PRCCISSCCRPSCCV | PRCCISSFCRPSCCV | KRTAP4-11 | 56 | 189 | 0 | 325 |
| PanTT26-P69 | CRPQCCQSVCCQPTC | CRPQCCQTVCCQPTC | KRTAP4-9 | 101 | 0 | 279 | 0 |
| PanTT26-P70 | TCCRTTCYRPSCCVS | TCCRTTCFRPSCCVS | KRTAP4-9 | 217 | 118 | 67 | 0 |
| PanTT26-P71 | PGESLRPRGERRLPQ | PGESLRPLGERRLPQ | LILRA6 | 37 | 0 | 0 | 0 |
| PanTT26-P72 | PGSGPQNRLGRYLEV | PGSGPQNGLGRYLEV | LILRB3 | 111 | 0 | 0 | 0 |
| PanTT26-P73 | ETGPEAERLEQLESG | ETGPEAEWLEQLESG | LOC642846 | 55 | 0 | 0 | 0 |
| PanTT26-P74 | QKEKSLEFTKELPGY | QKEKSLELTKELPGY | LRRC37A3 | 64 | 0 | 0 | 0 |
| PanTT26-P75 | PFSPSHPAPPSDPSH | PFSPSHPGPPSDPSH | MAGI2 | 90 | 24 | 0 | 0 |
| PanTT26-P76 | DEMDCPLSPTPPLCS | DEMDCPLRPTPPLCS | MALRD1 | 117 | 51 | 222 | 527 |
| PanTT26-P77 | VKDQGPMVSAPVKDQ | VKDQGPMFSAPVKDQ | MAP6 | 136 | 0 | 0 | 0 |
| PanTT26-P78 | KDQGPIVPAPVKGEG | KDQGPIVTAPVKGEG | MAP6 | 4 | 0 | 0 | 0 |
| PanTT26-P79 | TTTASTEGSETTTAS | TTTASTECSETTTAS | MUC22 | 42 | 0 | 0 | 0 |
| PanTT26-P80 | LRPQLAENKQQFRNL | LRPQLAEKKQQFRNL | NBPF1 | 75 | 125 | 0 | 0 |
| PanTT26-P81 | EKKQQFRNLKEKCFL | EKKQQFRSLKEKCFL | NBPF10 | 169 | 212 | 0 | 164 |
| PanTT26-P82 | AFMYAKKEEWKKAEE | AFMYAKKGEWKKAEE | NCF2 | 29 | 60 | 0 | 0 |
| PanTT26-P83 | KLKKKQVNVFA | KLKKKQVKVFA | NCOR1 | 106 | 327 | 0 | 710 |
| PanTT26-P84 | GRLILWEAPPLGAGG | GRLILWEGPPLGAGG | NEK8 | 97 | 32 | 0 | 164 |
| PanTT26-P85 | NQLKERSFAQLISKD | NQLKERSIAQLISKD | NLRP14 | 108 | 0 | 108 | 0 |
| PanTT26-P86 | ILLIHCDAHLHTPMY | ILLIHCDTHLHTPMY | OR2T4 | 61 | 35 | 0 | 0 |
| PanTT26-P87 | LLIHCDAHLHTPMYF | LLIHCDAYLHTPMYF | OR2T4 | 74 | 43 | 0 | 0 |
| PanTT26-P88 | AVVFQDSVVFRVAPW | AVVFQDSMVFRVAPW | PADI4 | 19 | 21 | 0 | 0 |
| PanTT26-P89 | EHSQETESLREALLS | EHSQETEILREALLS | PDE4DIP | 9 | 15 | 0 | 0 |
| PanTT26-P90 | PYGCLPTGDRTGLIE | PYGCLPTRDRTGLIE | PIK3CD | 9 | 0 | 0 | 0 |
| PanTT26-P91 | GLPTDTIRKEFRTRM | GLPTDTICKEFRTRM | PLCB4 | 81 | 93 | 0 | 20 |
| PanTT26-P92 | TGAMNVAKGTIQTGV | TGAMNVAIGTIQTGV | PLIN4 | 107 | 99 | 0 | 124 |
| PanTT26-P93 | TYSPTSPVYTPTSPK | TYSPTSPDYTPTSPK | POLR2A | 66 | 0 | 511 | 0 |
| PanTT26-P94 | CRGSGKSNVGTSGDH | CRGSGKSKVGTSGDH | POTEH | 7 | 41 | 0 | 0 |
| PanTT26-P95 | SKMGKWCRHCFAWCR | SKMGKWCSHCFAWCR | POTEH | 200 | 176 | 58 | 149 |
| PanTT26-P96 | SKMGKWCRHCFPCCR | SKMGKWCSHCFPCCR | POTEJ | 297 | 261 | 0 | 137 |
| PanTT26-P97 | GEPIPQPARLRYVTS | GEPIPQPVRLRYVTS | PRICKLE2 | 12 | 27 | 0 | 0 |
| PanTT26-P98 | VQLRGRAQGGGALRA | VQLRGRALGGGALRA | PRSS22 | 20 | 72 | 0 | 0 |
| PanTT26-P99 | NLVHGPPAPPQVGAD | NLVHGPPGPPQVGAD | PSD | 57 | 97 | 80 | 328 |
| PanTT26-P100 | GGGPDGPLYKVSVTA | GGGPDGPRYKVSVTA | RAP1GAP | 44 | 0 | 60 | 0 |
| PanTT26-P101 | GGHDSSSWSHRYGGG | GGHDSSSLSHRYGGG | RBMXL3 | 9 | 0 | 0 | 0 |
| PanTT26-P102 | VRRCLPLCALTLEAA | VRRCLPLWALTLEAA | RHCE | 9 | 75 | 0 | 0 |
| PanTT26-P103 | PSRHRYGARQPRARL | PSRHRYGTRQPRARL | RNF126 | 152 | 233 | 0 | 142 |
| PanTT26-P104 | ETKTKDEMAAAEEKV | ETKTKDETAAAEEKV | RNF213 | 0 | 0 | 0 | 0 |
| PanTT26-P105 | QEVEGETQKTEGDAQ | QEVEGETHKTEGDAQ | RP1L1 | 0 | 22 | 0 | 0 |
| PanTT26-P106 | KSEGEEAQEVEGETQ | KSEGEEAHEVEGETQ | RP1L1 | 3 | 29 | 0 | 105 |
| PanTT26-P107 | AGRFGQGAHHAAGQA | AGRFGQGDHHAAGQA | SBSN | 95 | 80 | 44 | 205 |
| PanTT26-P108 | QLLEGLGFTLTVVPE | QLLEGLGCTLTVVPE | SERP | 47 | 2 | 425 | 0 |
| | | | INA13P | | | | |
| PanTT26-P109 | TRLFPNEFANFYNAV | TRLFPNELANFYNAV | SLITRK1 | 47 | 46 | 0 | 0 |
| PanTT26-P110 | QQEIDQKRLEFEKQK | QQEIDQKKIRINAKT | SMC1B | 23 | 0 | 0 | 0 |
| PanTT26-P111 | KELRALRKMVSNMSG | KELRALREMVSNMSG | SPERT | 145 | 65 | 0 | 0 |
| PanTT26-P112 | AGQNPASHPPPDDAE | AGQNPASDPPPDDAE | TADA1 | 13 | 10 | 0 | 0 |
| PanTT26-P113 | PTKCEVERFTATSFG | PTKCEVEQFTATSFG | TG | 24 | 17 | 0 | 0 |
| PanTT26-P114 | IHSSWDCGLFTNYSA | IHSSWDCSLFTNYSA | TMC8 | 38 | 61 | 0 | 408 |
| PanTT26-P115 | IMASKGMRHFCLISE | IMASKGMHHFCLISE | TMEM168 | 132 | 92 | 213 | 193 |
| PanTT26-P116 | LWHLQGPKDLMLKLR | LWHLQGPEDLMLKLR | TMPRSS6 | 275 | 12 | 371 | 0 |
| PanTT26-P117 | GRNSFEVRVCACPGR | GRNSFEVLVCACPGR | TP53 | 92 | 42 | 0 | 0 |
| PanTT26-P118 | TSCARRDDPRASSPN | TSCARRDYPRASSPN | TRIOBP | 0 | 53 | 0 | 0 |
| PanTT26-P119 | LGLWRGEEVTLSNPK | LGLWRGEAVTLSNPK | TRIP12 | 13 | 74 | 0 | 240 |
| PanTT26-P120 | GCLGGENRFRLRLES | GCLGGENCFRLRLES | TRPM4 | 0 | 26 | 0 | 7 |
| PanTT26-P121 | TQLRLPGCPTPVSFG | TQLRLPGWPTPVSFG | VARS2 | 0 | 99 | 0 | 0 |
| PanTT26-P122 | RKFISLHRKALESDF | RKFISLHKKALESDF | WDFY4 | 23 | 249 | 0 | 562 |
| PanTT26-P123 | SGSGSGPLPSLFLNS | SGSGSGPFPSLFLNS | ZFHX3 | 76 | 13 | 85 | 65 |
| PanTT26-P124 | GCGKVFARSENLKIH | GCGKVFACSENLKIH | ZIC1 | 43 | 0 | 186 | 0 |
| PanTT26-P125 | STLLTEHRRIHTGEK | STLLTEHLRIHTGEK | ZNF135 | 0 | 10 | 0 | 0 |
| PanTT26-P126 | EKPYLCPDCGRGFGQ | EKPYLCPECGRGFGQ | ZNF169 | 0 | 0 | 0 | 0 |
| PanTT26-P127 | EECGKPFNRFSYLTV | EECGKPFKRFSYLTV | ZNF257 | 0 | 80 | 0 | 0 |
| PanTT26-P128 | YECNECGKAFSQSSH | YECNECGNAFSQSSH | ZNF3 | 0 | 0 | 0 | 0 |
| PanTT26-P129 | SHNSSLILHQRVHTG | SHNSSLIFHQRVHTG | ZNF304 | 0 | 67 | 0 | 0 |
| PanTT26-P130 | VTGGRGGRQGPSPAF | VTGGRGGWQGPSPAF | ZNF385C | 58 | 82 | 0 | 3 |
| PanTT26-P131 | CNFSTIDVVSLKTDT | CNFSTIDVSLKTDTE | ZNF407 | 56 | 40 | 0 | 70 |
| PanTT26-P132 | SNLTKHKKIHIEKKP | SNLTKHKIIHIEKKP | ZNF43 | 369 | 170 | 703 | 0 |
| PanTT26-P133 | ECGQAFSLSSNLMRH | ECGQAFSISSNLMRH | ZNF479 | 0 | 80 | 0 | 0 |
| PanTT26-P134 | IHKMIHTGEKPYKCE | IHKMIHTVEKPYKCE | ZNF493 | 0 | 38 | 19 | 0 |
| PanTT26-P135 | CNECGKAFCQSPSLI | CNECGKALCQSPSLI | ZNF501 | 0 | 36 | 0 | 0 |
| PanTT26-P136 | ECGKAFNRSSNLTKH | ECGKAFNSSSNLTKH | ZNF506 | 0 | 17 | 0 | 0 |
| PanTT26-P137 | LQNHIQTIHRELVPD | LQNHIQTFHRELVPD | ZNF521 | 94 | 71 | 0 | 0 |
| PanTT26-P138 | SNDSSLTQHQRVHTG | SNDSSLTHHQRVHTG | ZNF570 | 0 | 52 | 0 | 0 |
| PanTT26-P139 | SNLTTHKKIHTGERP | SNLTTHKIIHTGERP | ZNF626 | 66 | 24 | 14 | 109 |
| PanTT26-P140 | NVAKPSSGPHTLLHI | NVAKPSSCPHTLLHI | ZNF626 | 80 | 0 | 465 | 0 |
| PanTT26-P141 | STLNTHKRIHTGEEP | STLNTHKSIHTGEEP | ZNF679 | 0 | 21 | 0 | 0 |
| PanTT26-P142 | KCDECGNVFNWPATL | KCDECGNDFNWPATL | ZNF680 | 0 | 24 | 0 | 0 |
| PanTT26-P143 | CKECGKAFSSSSHLI | CKECGKALSSSSHLI | ZNF699 | 0 | 0 | 0 | 0 |
| PanTT26-P144 | HQRTHTGEKPFKCDE | HQRTHTGDKPFKCDE | ZNF7 | 0 | 38 | 0 | 0 |
| PanTT26-P145 | EECGKAFSVFSTLTK | EECGKAFRVFSTLTK | ZNF708 | 12 | 70 | 0 | 0 |
| PanTT26-P146 | HKRIHNGEKPYKCEE | HKRIHNGDKPYKCEE | ZNF730 | 0 | 137 | 0 | 298 |
| PanTT26-P147 | EKPYSCPDCSLRFAY | EKPYSCPECSLRFAY | ZNF785 | 94 | 15 | 159 | 0 |
| PanTT26-P148 | KCEECDTVFSRKSHH | KCEECDTDFSRKSHH | ZNF860 | 47 | 28 | 396 | 0 |
| PanTT26-P149 | KAFSQSSTLRKHEII | KAFSQSSSLRKHEII | ZNF99 | 0 | 43 | 0 | 0 |

For example, increased IFN-γ production to the mutated KRAS peptide KLWVGAVGVGKSAL was observed after 3× stimulation with the autologous tumor cell-stimulated TILs compared to young TILs (Table 1). The clinical relevance of this finding is underlined by the established knowledge that oncogenic mutant KRAS commonly plays a crucial role in PDAC pathogenesis (Eser et al, 2014). The strongest IFN-γ response by both PanTT26 young TILs and tumor cell-stimulated TILs was observed after exposure to a mutated peptide from NCOR1 (KLKKKQVKVFA, mutation: N99K). Young TILs produced 327 pg IFN-γ/10e5 TILs in response to mutated NCOR1, while tumor cell-stimulated TILs showed 710 pg IFN-γ/10e5 TIL (Table 1).

PanTT26 TILs also showed strong IFN-γ responses to a mutated peptide derived from WDFY4 (RKFISLHKKALESDF), which is a protein likely associated with autoimmune diseases such as systemic lupus erythematosus and rheumatoid arthritis. 17% of mutations (25/149 mutations) in PanTT26 are associated with zinc finger proteins (ZNF), which display diverse biological functions (Cassandri et al, 2017). The recognition of a ZNF730-derived peptide was pronounced following stimulation of PanTT26 TILs with autologous tumor cells, although four other wild type ZNF peptides were recognized (Table 1). It is plausible that a high number of wild type ZNF targets were obtained due to the filter that was applied for detecting mutations in the tumor samples (minimum of 5% mutation load). However, the function and immunological significance of ZNF as a target for cellular immune responses in pancreatic cancer therefore warrants further exploration.

Patient Pan TT39

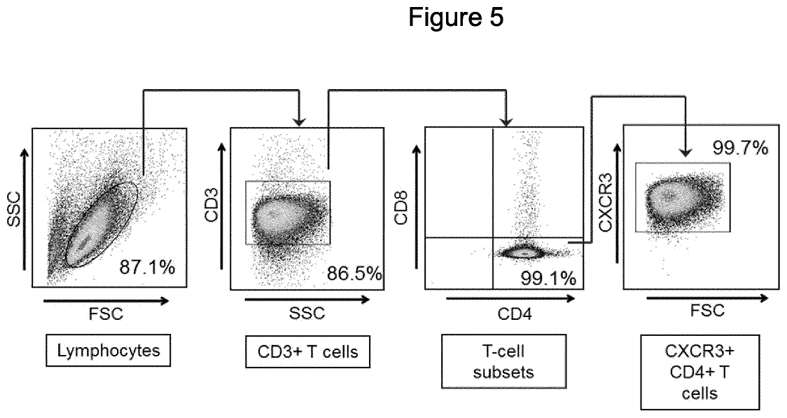

TILs isolated from this patient were characterized by flow cytometry and found to contain exclusively CD4⁺ T-cells (>99%) (FIG. 5). Whole-exome sequencing was performed using DNA for PanTT39 tumor tissue revealing mutated as well as the corresponding wild type peptide sequences to gauge for T-cell reactivity. Following mutation analysis, 1447 mutations were found, as compared to 149 mutations in PanTT26 tumor, thus reflecting a 10-fold higher mutational burden in patient PanTT39. A mutation in the BRCA1 gene product (R600L) was also identified. This is of note, since BRCA1 mutations are implicated as a key contributing factor related to the burden of somatic mutations in pancreatic cancer (Waddell et al, 2015). Seven point mutations in the HLA-A alleles were found, two point mutations in the HLA-B alleles and eight point mutations in the HLA-C alleles, which ultimately gave rise to amino acid changes in the resulting protein products associated with the HLA class I antigen processing and presentation pathway (Table 2).

TABLE 2
List of mutations in HLA class I
and II molecules identified by
whole-exome sequencing of tumor
tissue from patient PanTT26
and PanTT39

| | | Location | | |
|---|---|---|---|---|
| | | of | | |
| | HLA | mutation | Wild | |
| Patient | class/ | in | type | Mutated |
| ID | type | protein | sequence | sequence |
| PanTT26 | HLA- | A102G | DILEQARAA | DILEQARGAV |
| | DRB1 | | VDTYCR | DTYCR |
| | HLA-A | G10W | MYGCDVGSD | MYGCDVGSDW |
| | | | GRFLRGYRQ | RFLRGYRQDA |
| | | | DAYD | YD |
| | HLA-A | G131W | IQIMYGCDV | IQIMYGCDVG |
| | | | GPDGRFLRG | PDWRFLRGYR |
| | | | YRQDAYD | QDAYD |
| PanTT39 | HLA-A | G131W | IQIMYGCDV | IQIMYGCDVG |
| | | | GSDGRFLRG | SDWRFLRGYR |
| | | | YRQDAYD | QDAYD |
| | HLA-A | R138Q | DVGPDGRFL | DVGPDGRFLR |
| | | | RGYRQDAYD | GYQQDAYDGK |
| | | | GKDYIAL | DYIAL |
| | HLA-A | R138Q | DVGSDGRFL | DVGSDGRFLR |
| | | | RGYRQDAYD | GYQQDAYDGK |
| | | | GKDYIAL | DYIAL |
| | HLA-A | D140Y | GSDGRFLRG | GSDGRFLRGY |
| | | | YRQDAYDGK | RQYAYDGKDY |
| | | | DYIALNE | IALNE |
| | HLA-A | D140Y | GPDGRFLRG | GPDGRFLRGY |
| | | | YRQDAYDGK | RQYAYDGKDY |
| | | | DYIALNE | IALNE |
| | HLA-B | Y167H | YLEGECVEW | YLEGECVEWL |
| | | | LRRYLENGK | RRHLENGKDK |
| | | | DKLERAG | LERAG |
| | HLA-B | Y195H | YLEGECVEW | YLEGECVEWL |
| | | | LRRYLENGK | RRHLENGKDK |
| | | | DKLERAD | LERAD |
| | HLA-C | R30K | ALTETWACS | ALTETWACSH |
| | | | HSMRYFDTA | SMKYFDTAVS |
| | | | VSRPGRG | RPGRG |
| | HLA-C | R2K | MRYFDTAVS | MKYFDTAVSR |
| | | | RPGRG | PGRG |
| | HLA-C | D5V | MRYFDTAVS | MRYFVTAVSR |
| | | | RPGRGEPR | PGRGEPR |
| | HLA-C | D5Y | MRYFDTAVS | MRYFYTAVSR |
| | | | RPGRGEPR | PGRGEPR |
| | HLA-C | A7S | MRYFDTAVS | MRYFDTSVSR |
| | | | RPGRGEPRFI | PGRGEPRFI |
| | HLA-C | D33Y | ETWACSHSM | ETWACSHSMR |
| | | | RYFDTAVSR | YFYTAVSRPG |
| | | | PGRGEPR | RGEPR |
| | HLA-C | D33V | ETWACSHSM | ETWACSHSMR |
| | | | RYFDTAVSR | YFVTAVSRPG |
| | | | PGRGEPR | RGEPR |
| | HLA-C | A35S | WACSHSMRY | WACSHSMRYF |
| | | | FDTAVSRPG | DTSVSRPGRG |
| | | | RGEPRFI | EPRFI |
| | HLA- | 192R | FRNQKGHSG | FRNQKGHSGL |
| | DRB1 | | LQPTGNTF | QPRGNTF |
| | HLA- | 1262R | FRNQKGHSG | FRNQKGHSGL |
| | DRB1 | | LQPTGFLS | QPRGFLS |
| | HLA- | A42V | GAIKADHVS | GAIKADHVST |
| | DPA1 | | TYAAFVQTH | YAVFVQTHRP |
| | | | RPTGEFM | TGEFM |
| | HLA- | A421 | GAIKADHVS | GAIKADHVST |
| | DPA1 | | TYAAFVQTH | YATFVQTHRP |
| | | | RPTGEFM | TGEFM |

WT = wild type; Mut = mutated

Since the TILs from PanTT39 consisted exclusively of CD4⁺ T-cells and no CD8⁺ T-cells, the analysis was focused on the peptides that could bind HLA class II molecules. In Table 3, fourteen HLA class II-binding targets are shown that were identified using a predicted consensus rank of less than or equal to 1.0. It is important to mention that the mutational burden among HLA-DRB1 alleles in PanTT39 tumor was calculated as 8.8%. Peptides that would bind to HLA-DRB1 were nevertheless incorporated, assuming >90% chance that an adequate number of tumor cells would still be able to present antigen via HLA-DRB1. TILs from this patient were then screened for recognition of peptides in a three-day 96-well co-culture assay, as described for PanTT26 TILs.

TABLE 3
List of predicted HLA class II-binding
peptides for stimulation assays with
TILs from patient PanTT39

| | | | | HLA class |
|---|---|---|---|---|
| Peptide | | Sequence | | II re- |
| ID | | (wild | Sequence | stricting |
| name | Gene | type) | (Mutated) | element |
| PanTT39- | DOCK3 | AFTLLLYC | AFTMLLYCE | HLA- |
| P1 | | ELLQWED | LLQWED | DQA10101- |
| | | | | DQB10201 |
| PanTT39- | AQP7 | TIYSLFYS | TIYSLFYSV | HLA- |
| P2 | | VADRDAPA | ADQDAPA | DQA10501- |
| | | | | DQB10501 |
| PanTT39- | CCDC39 | FKQDLMI | FKQDLML | HLA- |
| P3 | | EDNLL | EDNLL | DQA10101- |
| | | | | DQB10201 |
| PanTT39- | CCDC39 | QDLMIED | QDLMLEDN | DRB1-0301 |
| P4 | | NLLKLEV | LLKLEV | |
| PanTT39- | K7N7A8 | GLLRDW | GLLRYWRT | DRB1-0301 |
| P5 | | RTERLF | ERLF | |
| PanTT39- | K7N7A8 | ILFSLQP | ILFSLQPG | DRB1-0101 |
| P6 | | GLLRDW | LLRYW | |
| PanTT39- | TENM3 | NVSFFH | NVSFFH | HLA- |
| P7 | | YPEYGY | YQEYGY | DQA10101- |
| | | | | DQB10501 |
| PanTT39- | IPO8 | EFPVRQA | EFPVLQA | DRB1-0101 |
| P8 | | AAIYLK | AAIYLK | |
| PanTT39- | CFTR | INFKIER | INFKIER | DRB1-0101 |
| P9 | | GQLLAV | GQLAV | |
| PanTT39- | VPS4B | GAIVIER | GAIVIEL | DRB1-0301 |
| P10 | | PNVKWS | PNVKWS | |
| PanTT39- | MORC1 | LNKVTIDA | LNKVTIDA | DRB1-0301 |
| P11 | | RHRLPL | IHRLPL | |
| PanTT39- | CDKL3 | DFGFARTL | DFGFALT | DRB1-0101 |
| P12 | | AAPGDI | LAAPGDI | |
| PanTT39- | NUP93 | LELMNKLL | LELINKL | DRB1-0101 |
| P13 | | SPVVPQ | LSPVVPQ | |
| PanTT39- | SPTA1 | IEELRHLW | IEELHHLW | HLA- |
| P14 | | DLLLELTL | DLLLELTL | DQA10101- |
| | | | | DQB10201 |

Table 4 shows that PanTT39 TILs produced lower IFN-γ/10⁵ TIL in response to mutated peptides as compared to PanTT26 TILs. It is considered the possibility that CD4⁺ T-cells in PanTT39 TILs could comprise a mixture of different T-cell subsets, e.g. Th1, Th2 and Th17.

TABLE 4
Antigen-specific IFN-γ production to
mutated and the corresponding wild type
target by ‘young’ TILs from patient
PanTT39.
Legend: WT = wild type; Mut = mutant.

| | | | | IFN-γ | |
|---|---|---|---|---|---|
| | | | | (pg/10⁵ | |
| | | | | TIL/ | |
| | | | | 1 microgram | |
| Peptide | Wild type | Mutated | Gene | peptide | |
| ID | sequence | sequence | Name | WT | Mut |
| PanTT39-P1 | AFTLLLYC | AFTMLLYC | DOCK3 | 29 | 28 |
| | ELLQWED | ELLQWED | | | |
| PanTT39-P2 | TIYSLFYS | TIYSLFYS | AQP7 | 21 | 1 |
| | VADRDAPA | VADQDAPA | | | |
| PanTT39-P3 | FKQDLMIE | FKQDLML | CCDC39 | 4 | 0 |
| | DNLL | EDNLL | | | |
| PanTT39-P4 | QDLMIEDN | QDLMLED | CCDC39 | 0 | 11 |
| | LLKLEV | NLLKLEV | | | |
| PanTT39-P5 | GLLRDWR | GLLRYW | K7N7A8 | 0 | 6 |
| | TERLF | RTERLF | | | |
| PanTT39-P6 | ILFSLQP | ILFSLQ | K7N7A8 | 21 | 6 |
| | GLLRDW | PGLLRYW | | | |
| PanTT39-P7 | NVSFFHY | NVSFFH | TENM3 | 16 | 17 |
| | PEYGY | YQEYGY | | | |
| PanTT39-P8 | EFPVRQA | EFPVLQA | IPO8 | 14 | 10 |
| | AAIYLK | AAIYLK | | | |
| PanTT39-P9 | INFKIER | INFKIER | CFTR | 6 | 0 |
| | GQLLAV | GQLAV | | | |
| PanTT39-P10 | GAIVIER | GAIVIEL | VPS4B | 6 | 2 |
| | PNVKWS | PNVKWS | | | |
| PanTT39-P11 | LNKVTID | LNKVTID | MORC1 | 9 | 14 |
| | ARHRLPL | AIHRLPL | | | |
| PanTT39-P12 | DFGFART | DFGFALT | CDKL3 | 11 | 6 |
| | LAAPGDI | LAAPGDI | | | |
| PanTT39-P13 | LELMNKL | LELINKL | NUP93 | 0 | 19 |
| | LSPVVPQ | LSPVVPQ | | | |
| PanTT39-P14 | IEELRHLW | IEELHHLW | SPTA1 | 16 | 0 |
| | DLLLELTL | DLLLELTL | | | |

In order to better define TIL PanTT39 reactivity, a T-cell product was obtained by cultivation of the TILs as described above. Flow cytometry analysis revealed that the the T-cell product was positive for TCR Vβ9⁺ (see FIG. 2A). To test if the T-cell product was able to recognize any of the mutated peptides tested earlier in the screening assay the cells were co-incubated with the same panel of HLA class II-binding peptides for three days, after which IFN-γ production in the supernatant was detected by ELISA. A single mutated peptide was strongly recognized by T-cell product, namely GLLRYWRTERLF (wild type sequence: GLLRDWRTERLF), which derives from an uncharacterized protein product of 449 amino acids encoded by the K7N7A8 gene. The T-cell product produced a cytotoxic response against the autologous tumor cell line that was assessed in a standard CD107a induction assay and which result is illustrated in FIG. 2A. In addition, the T-cell product also produced 480 mg/ml IFN-γ in response to GLLRYWRTERLF, compared to a meagre 6 pg IFN-γ/10⁵ TIL by the TILs before 3× stimulation with autologous tumor cells. Reactivity of T-cell product to the mutated peptide GLLRYWRTERLF could be blocked with the L243 antibody (anti-HLA class-II, DR) in a dose-dependent manner (see FIG. 2B). No difference in peptide reactivity of the T-cell product was observed in the presence of the W6/32 antibody (anti-HLA-I), further affirming that GLLRYWRTERLF contained a nominal HLA class II neoepitope. Using peptide titration, it was observed that the GLLRYWRTERLF mutated peptide induced robust IFN-γ production by the T-cell product from patient PanTT39 even at low peptide concentrations, indicating the presence of high-affinity TCRs (see FIG. 2C). The wild type peptide was also able to activate T-cells at concentrations at the high concentration of 5 μg peptide/well.

Patient Pan TT77

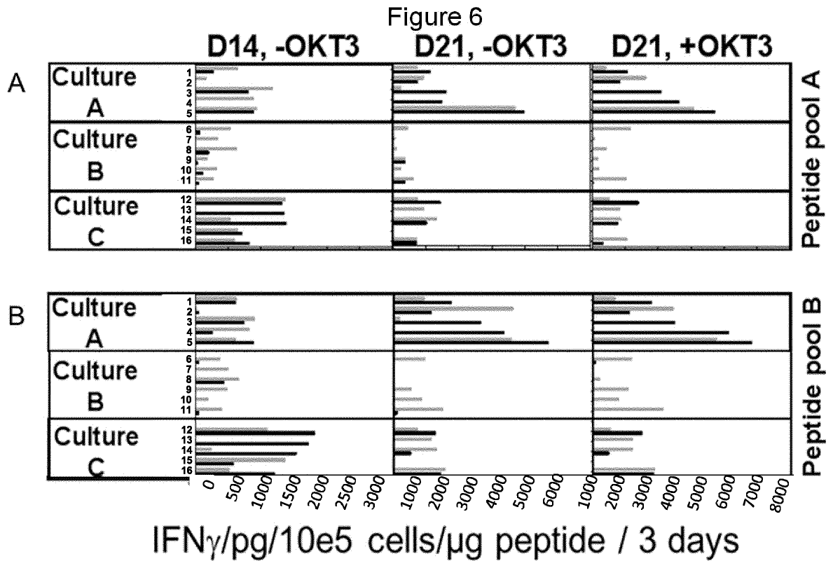

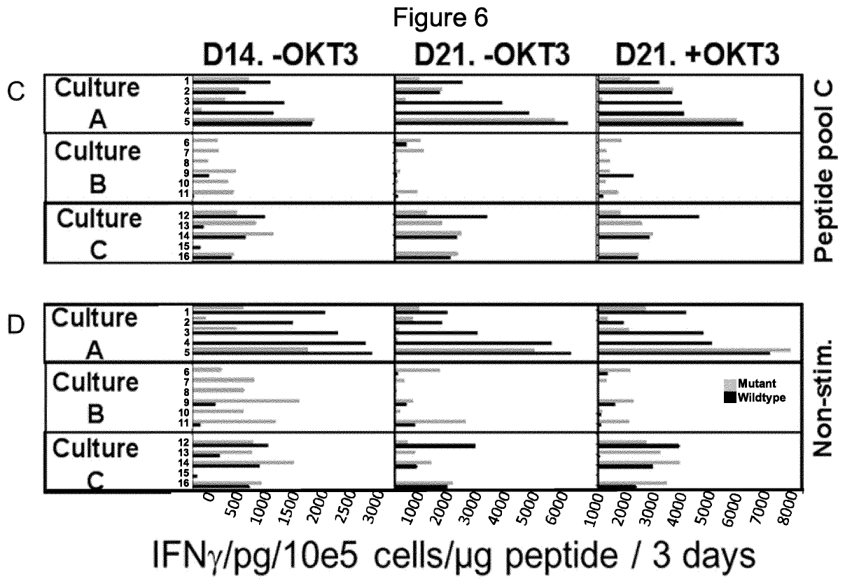

FIG. 3A shows that PanTT77 TILs comprised approximately 84% CD4⁺ T-cells and 14% CD8⁺ T-cells. Immunoreactivity of PBMCs as well as TILs from this patient to a panel of mutant and wild type peptide sequences was assessed. In FIG. 3B, the unique peptide recognition profile marked by IFN-γ production was observed in PBMCs (five mutated peptides) and in TILs (nine mutated peptides), showing PBMCs from patient PanTT77 had a rather broad recognition of private neoepitopes without in vitro re-stimulation. FIG. 6 shows that a set of mutant peptides were only recognized by TILs e.g. the Protein Phosphatase 1 Regulatory Subunit 15B (PPP1R15B), which is part of an enzyme that dephosphorylates the eukaryotic translation initiation factor 2A (involved in regulating RNA translation into proteins) in response to stress, and with pro-oncogenic characteristics in breast cancer (Shahmoradgoli et al, 2013); neurobeachin-like protein 1 (NBEAL1), a protein that is expressed in the brain, testes and kidneys but overexpressed in gliomas (Chen et al, 2004); Ankyrin Repeat And Sterile Alpha Motif Domain Containing 1B (ANKS1B), which is expressed in normal brain tissue and is required for development, but also implicated in the pathogenesis of Alzheimer's Disease and downregulated in smoking-related clear-cell renal cell carcinoma (Eckel-Passow et al, 2014; Ghersi et al, 2004); Ciliogenesis Associated TTC17 Interacting Protein (CATIP/C2orf62), a protein involved in cilium biogenesis by inducing actin polymerization (Bontems et al, 2014); Calcium Voltage-Gated Channel Subunit Alpha1 S (CACNA1S), a subunit of a voltage-gated calcium channel with an important role in interacting with the ryanodine receptor in muscle cells for excitation-contraction coupling (Wu et al, 2015).

As shown in FIGS. 3B and 6, some of the mutated peptides were recognized by TILs and PBMCs (six mutated peptides) and triggered stronger IFN-γ production in PBMCs (up to IFN-γ 350 pg/10⁵ PBMCs) compared to TILs (up to 141 μg IFN-γ/10⁵ TIL). A single mutated peptide, derived from the Proline Rich Transmembrane Protein 1 (PRRT1, also known as SynDIG4), and induced strong IFN-γ by PBMCs and TILs. PRRT1 is part of the α-amino-3-hydroxy-5-methyl-4-isoxazolepropionic acid receptor (AMPAR) complex, which is involved in glutamate transport in the central nervous system and is important for synaptic transmission (Kirk et al, 2016; von Engelhardt et al, 2010). No mutations were found in the HLA class I and class II pathways in this patient's tumor.

Table 5 shows the IFN-γ production of PBMC T-cells co-cultured and stimulated with neoepitopes that trigger a response in TIL T-cells but not PBMCs in an initial stimulation step. PBMC T-cells were co-cultured one or two times one of the peptides in the presence or absence of OKT3 and then tested for IFN-γ production by stimulation using the respective peptide. As can be seen in Table 5, all six neoepitopes were recognized by the PBMC T-cells already after the first co-cultivation. Interestingly, a second co-cultivation step led to no recognition of the WT epitope and increased IFN-γ production upon stimulation with the mutated epitope in the absence of OKT3. The response increases further when co-cultivation was performed in the presence of OKT3.

TABLE 5
IFN-γ production by PanTT77 PBMCs
to private mutated targets (and the
corresponding wild type sequences)
after a first co-cultivation with
stimulating peptides without OKT3
and after a second co-cultivation
wit stimulating peptides
with and without OKT3.
Legend: WT = wild type;
Mut = mutant

| | | | After | | After | | After | |
|---|---|---|---|---|---|---|---|---|
| | | | first | | second | | second | |
| | | | co- | | co- | | co- | |
| | | | culti- | | culti- | | culti- | |
| Wild | | | vation | | vation | | vation | |
| type | Mutated | Gene | (−OKT3) | | (−OKT3) | | (+OKT3) | |
| sequence | sequence | Name | WT | Mut | WT | Mut | WT | Mut |
| PLSQESE | PLSQESE | VCX3A | 50 | 358 | 83 | 1451 | 0 | 2822 |
| VEEPLSQE | MEEPLSQE | | | | | | | |
| GFQTLTP | GFQTLTP | PPP1R15B | 0 | 489 | 0 | 838 | 0 | 1035 |
| ESSCLRED | DSSCLRED | | | | | | | |
| EPFTTLH | EPFTTLH | NBEAL1 | 426 | 643 | 0 | 516 | 0 | 1434 |
| IQLQSGRF | TQLQSGRF | | | | | | | |
| DDLSRQD | DDLSRQD | ANKS1B | 0 | 469 | 0 | 0 | 0 | 281 |
| DNDPPKEY | GNDPPKEY | | | | | | | |
| FIVEQTV | FIVEQTV | C2orf62 | 0 | 183 | 0 | 0 | 0 | 11 |
| HAEEGIPM | QAEEGIPM | | | | | | | |
| LAVYLPM | LAVYLPM | CACNA1S | 44 | 392 | 0 | 918 | 107 | 1553 |
| PEDDNNSL | SEDDNNSL | | | | | | | |

### REFERENCES

Antonioli L, Yegutkin G G, Pacher P, Blandizzi C, Haskó G (2016) Anti-CD73 in cancer immunotherapy: awakening new opportunities. Trends in cancer 2(2): 95-109
Benson A, Olsen J, Sasson A (2016) Pancreatic, Neuroendocrine G I, and Adrenal Cancers. In Cancer Management: A Multidisciplinary Approach, Haller D, Wagman L, Camphausen K, Hoskins W (eds): Cancer Network
Bonaz B, Sinniger V, Pellissier S (2017) The Vagus Nerve in the Neuro-Immune Axis: Implications in the Pathology of the Gastrointestinal Tract. Frontiers in immunology 8: 1452
Bontems F, Fish R J, Borlat I, Lembo F, Chocu S, Chalmel F, Borg J P, Pineau C, Neerman-Arbez M, Bairoch A, Lane L (2014) C2orf62 and TTC17 are involved in actin organization and ciliogenesis in zebrafish and human. PloS one 9(1): e86476
Brandes M, Willimann K, Moser B (2005) Professional antigen-presentation function by human gammadelta T Cells. Science 309(5732): 264-8
Cao H, Le D, Yang L X (2013) Current status in chemotherapy for advanced pancreatic adenocarcinoma. Anticancer research 33(5): 1785-91
Carreno B M, Magrini V, Becker-Hapak M, Kaabinejadian S, Hundal J, Petti A A, Ly A, Lie W R, Hildebrand W H, Mardis E R, Linette G P (2015) Cancer immunotherapy. A dendritic cell vaccine increases the breadth and diversity of melanoma neoantigen-specific T-cells. Science 348(6236): 803-8
Cassandri M, Smirnov A, Novelli F, Pitolli C, Agostini M, Malewicz M, Melino G, Raschella G (2017) Zinc-finger proteins in health and disease. Cell Death Discov 3: 17071
Chen J, Lu Y, Xu J, Huang Y, Cheng H, Hu G, Luo C, Lou M, Cao G, Xie Y, Ying K (2004) Identification and characterization of NBEAL1, a novel human neurobeachin-like 1 protein gene from fetal brain, which is up regulated in glioma. Brain research Molecular brain research 125(1-2): 147-55
Cohen C J, Gartner J J, Horovitz-Fried M, Shamalov K, Trebska-McGowan K, Bliskovsky W, Parkhurst M R, Ankri C, Prickett T D, Crystal J S, Li Y F, E I-Gamil M, Rosenberg S A, Robbins P F (2015) Isolation of neoantigen-specific T-cells from tumor and peripheral lymphocytes. The Journal of clinical investigation 125(10): 3981-91
Concha-Benavente F, Srivastava R, Ferrone S, Ferris R L (2016) Immunological and clinical significance of HLA class I antigen processing machinery component defects in malignant cells. Oral Oncol 58: 52-8
Conroy T, Desseigne F, Ychou M, Bouche O, Guimbaud R, Becouarn Y, Adenis A, Raoul J L, Gourgou-Bourgade S, de la Fouchardiere C, Bennouna J, Bachet J B, Khemissa-Akouz F, Pere-Verge D, Delbaldo C, Assenat E, Chauffert B, Michel P, Montoto-Grillot C, Ducreux M, Groupe Tumeurs Digestives of U, Intergroup P (2011) FOLFIRINOX versus gemcitabine for metastatic pancreatic cancer. The New England journal of medicine 364(19): 1817-25
Eckel-Passow J E, Serie D J, Bot B M, Joseph R W, Cheville J C, Parker A S (2014) ANKS1B is a smoking-related molecular alteration in clear cell renal cell carcinoma. BMC urology 14: 14
Eil R, Vodnala S K, Clever D, Klebanoff C A, Sukumar M, Pan J H, Palmer D C, Gros A, Yamamoto T N, Patel S J, Guittard G C, Yu Z, Carbonaro V, Okkenhaug K, Schrump D S, Linehan W M, Roychoudhuri R, Restifo N P (2016) Ionic immune suppression within the tumor microenvironment limits T-cell effector function. Nature 537: 539
Eil R L, Roychoudhuri R, Clever D, Patel S, Sukumar M, Pan J H, Palmer D, Klebanoff C A, Restifo N P (2015) Elevated potassium levels suppress T-cell activation within tumors. Journal for immunotherapy of cancer 3(Suppl 2): P403-P403
Eser S, Schnieke A, Schneider G, Saur D (2014) Oncogenic KRAS signalling in pancreatic cancer. British journal of cancer 111(5): 817-22
Garcia-Silva S, Aranda A (2004) The thyroid hormone receptor is a suppressor of ras-mediated transcription, proliferation, and transformation. Molecular and cellular biology 24(17): 7514-23
Ghersi E, Vito P, Lopez P, Abdallah M, D'Adamio L (2004) The intracellular localization of amyloid beta protein precursor (AbetaPP) intracellular domain associated protein-1 (AIDA-1) is regulated by AbetaPP and alternative splicing. Journal of Alzheimer's disease: JAD 6(1): 67-78
Gourgou-Bourgade S, Bascoul-Mollevi C, Desseigne F, Ychou M, Bouche O, Guimbaud R, Becouarn Y, Adenis A, Raoul J L, Boige V, Berille J, Conroy T (2013) Impact of FOLFIRINOX compared with gemcitabine on quality of life in patients with metastatic pancreatic cancer: results from the PRODIGE 4/ACCORD 11 randomized trial. Journal of clinical oncology: official journal of the American Society of Clinical Oncology 31(1): 23-9
Greening D W, Gopal S K, Xu R, Simpson R J, Chen W (2015) Exosomes and their roles in immune regulation and cancer. Seminars in cell & developmental biology 40: 72-81
Gros A, Parkhurst M R, Tran E, Pasetto A, Robbins P F, Ilyas S, Prickett T D, Gartner J J, Crystal J S, Roberts I M, Trebska-McGowan K, Wunderlich J R, Yang J C, Rosenberg S A (2016) Prospective identification of neoantigen-specific lymphocytes in the peripheral blood of melanoma patients. Nature medicine
Gros A, Robbins P F, Yao X, Li Y F, Turcotte S, Tran E, Wunderlich J R, Mixon A, Farid S, Dudley M E, Hanada K, Almeida J R, Darko S, Douek D C, Yang J C, Rosenberg S A (2014) PD-1 identifies the patient-specific CD8(+) tumor-reactive repertoire infiltrating human tumors. The Journal of clinical investigation 124(5): 2246-59
Gubin M M, Artyomov M N, Mardis E R, Schreiber R D (2015) Tumor neoantigens: building a framework for personalized cancer immunotherapy. The Journal of clinical investigation: 1-9
Heylmann D, Bauer M, Becker H, van Gool S, Bacher N, Steinbrink K, Kaina B (2013) Human CD4⁺CD25⁺ regulatory T-cells are sensitive to low dose cyclophosphamide: implications for the immune response. PloS one 8(12): e83384
Hiraoka N, Ino Y, Yamazaki-Itoh R, Kanai Y, Kosuge T, Shimada K (2015) Intratumoral tertiary lymphoid organ is a favourable prognosticator in patients with pancreatic cancer. British journal of cancer 112(11): 1782-90
Huang X, Jan L Y (2014) Targeting potassium channels in cancer. The Journal of cell biology 206(2): 151-162
Jones S, Wang T L, Shih Ie M, Mao T L, Nakayama K, Roden R, Glas R, Slamon D, Diaz L A, Jr., Vogelstein B, Kinzler K W, Velculescu V E, Papadopoulos N (2010) Frequent mutations of chromatin remodeling gene ARID1A in ovarian clear cell carcinoma. Science 330(6001): 228-31
Keck S, Schmaler M, Ganter S, Wyss L, Oberle S, Huseby E S, Zehn D, King C G (2014) Antigen affinity and antigen dose exert distinct influences on CD4 T-cell differentiation. Proceedings of the National Academy of Sciences of the United States of America 111(41): 14852-7
Kirk L M, Ti S W, Bishop H I, Orozco-Llamas M, Pham M, Trimmer J S, Diaz E (2016) Distribution of the SynDIG4/proline-rich transmembrane protein 1 in rat brain. The Journal of comparative neurology 524(11): 2266-80
Kowalewski D J, Stevanovic S, Rammensee H G, Stickel J S (2015) Antileukemia T-cell responses in CLL—We don't need no aberration. Oncoimmunology 4(7): e1011527
Liu Z, meng Q, Bartek J, Poiret T, Persson O, Rane L, Rangelova E, lilies C, Peredo I, Luo X, Rao M, Axelsson-Robertson R, Dodoo E, Maeurer M (2016) Tumor-infiltrating T-cells (TIL) from patients with glioma. Oncoimmunology
Loonen A J, Knoers N V, van Os C H, Deen P M (2008) Aquaporin 2 mutations in nephrogenic diabetes insipidus. Seminars in nephrology 28(3): 252-65
Lotze M T, Chang A E, Seipp C A, Simpson C, Vetto J T, Rosenberg S A (1986) High-dose recombinant interleukin 2 in the treatment of patients with disseminated cancer. Responses, treatment-related morbidity, and histologic findings. Jama 256(22): 3117-24
Lu Y C, Robbins P F (2016a) Cancer immunotherapy targeting neoantigens. Seminars in immunology 28(1): 22-7
Lu Y C, Robbins P F (2016b) Targeting neoantigens for cancer immunotherapy. Int Immunol 28(7): 365-70
Maeurer M J, Gollin S M, Martin D, Swaney W, Bryant J, Castelli C, Robbins P, Parmiani G, Storkus W J, Lotze M T (1996) Tumor escape from immune recognition: lethal recurrent melanoma in a patient associated with downregulation of the peptide transporter protein TAP-1 and loss of expression of the immunodominant MART-1/Melan-A antigen. The Journal of clinical investigation 98(7): 1633-41 Martinez-Iglesias O A, Alonso-Merino E, Gomez-Rey S, Velasco-Martin J P, Martin Orozco R, Luengo E, Garcia Martin R, Ibanez de Caceres I, Fernandez A F, Fraga M F, Gonzalez-Peramato P, Varona C, Palacios J, Regadera J, Aranda A (2016) Autoregulatory loop of nuclear corepressor 1 expression controls invasion, tumor growth, and metastasis. Proceedings of the National Academy of Sciences of the United States of America 113(3): E328-37
Mayer-Barber K D, Andrade B B, Oland S D, Amaral E P, Barber D L, Gonzales J, Derrick S C, Shi R, Kumar N P, Wei W, Yuan X, Zhang G, Cai Y, Babu S, Catalfamo M, Salazar A M, Via L E, Barry C E, 3rd, Sher A (2014) Host-directed therapy of tuberculosis based on interleukin-1 and type I interferon crosstalk. Nature 511(7507): 99-103
McGranahan N, Furness A J, Rosenthal R, Ramskov S, Lyngaa R, Saini S K, Jamal-Hanjani M, Wilson G A, Birkbak N J, Hiley C T, Watkins T B, Shafi S, Murugaesu N, Mitter R, Akarca A U, Linares J, Marafioti T, Henry J Y, Van Allen E M, Miao D, Schilling B, Schadendorf D, Garraway L A, Makarov V, Rizvi N A, Snyder A, Hellmann M D, Merghoub T, Wolchok J D, Shukla S A, Wu C J, Peggs K S, Chan T A, Hadrup S R, Quezada S A, Swanton C (2016) Clonal neoantigens elicit T-cell immunoreactivity and sensitivity to immune checkpoint blockade. Science 351(6280): 1463-9
Meng Q, Liu Z, Rangelova E, Poiret T, Ambati A, Rane L, Xie S, Verbeke C, Dodoo E, Del Chiaro M, Lohr M, Segersvard R, Maeurer M J (2016) Expansion of Tumor-reactive T Cells From Patients With Pancreatic Cancer. Journal of immunotherapy 39(2): 81-9
Meyers E E, Kronemberger A, Lira V, Rahmouni K, Stauss H M (2016) Contrasting effects of afferent and efferent vagal nerve stimulation on insulin secretion and blood glucose regulation. Physiological Reports 4(4): e12718
Principe D R, DeCant B, Mascariñas E, Wayne E A, Diaz A M, Diaz N, Hwang R, Pasche B, Dawson D W, Fang D, Bentrem D J, Munshi H G, Jung B, Grippo P J (2016) TGFβ signaling in the pancreatic tumor microenvironment promotes fibrosis and immune evasion to facilitate tumorigenesis. Cancer research 76(9): 2525-2539
Rizvi N A, Hellmann M D, Snyder A, Kvistborg P, Makarov V, Havel J J, Lee W, Yuan J, Wong P, Ho T S, Miller M L, Rekhtman N, Moreira A L, Ibrahim F, Bruggeman C, Gasmi B, Zappasodi R, Maeda Y, Sander C, Garon E B, Merghoub T, Wolchok J D, Schumacher T N, Chan T A (2015) Cancer immunology. Mutational landscape determines sensitivity to PD-1 blockade in non-small cell lung cancer. Science 348(6230): 124-8
Rosenberg S A, Lotze M T, Muul L M, Leitman S, Chang A E, Ettinghausen S E, Matory Y L, Skibber J M, Shiloni E, Vetto J T, et al. (1985) Observations on the systemic administration of autologous lymphokine-activated killer cells and recombinant interleukin-2 to patients with metastatic cancer. The New England journal of medicine 313(23): 1485-92
Rosenberg S A, Packard B S, Aebersold P M, Solomon D, Topalian S L, Toy S T, Simon P, Lotze M T, Yang J C, Seipp C A, et al. (1988) Use of tumor-infiltrating lymphocytes and interleukin-2 in the immunotherapy of patients with metastatic melanoma. A preliminary report. The New England journal of medicine 319(25): 1676-80 Rosenberg S A, Restifo N P (2015) Adoptive cell transfer as personalized immunotherapy for human cancer. Science 348(6230): 62-8
Sautes-Fridman C, Lawand M, Giraldo N A, Kaplon H, Germain C, Fridman W H, Dieu-Nosjean M C (2016) Tertiary Lymphoid Structures in Cancers: Prognostic Value, Regulation, and Manipulation for Therapeutic Intervention. Frontiers in immunology 7: 407
Schumacher T N, Schreiber R D (2015) Neoantigens in cancer immunotherapy. Science 348(6230): 69-74
Shahmoradgoli M, Riazalhosseini Y, Haag D, Becker N, Hovestadt V, Heck S, Sinn H P, Schneeweiss A, Mannherz O, Sahin O, Lichter P (2013) Protein phosphatase 1, regulatory subunit 15B is a survival factor for ERalpha-positive breast cancer. Int J Cancer 132(11): 2714-9
Shen W, Tao G-q, Zhang Y, Cai B, Sun J, Tian Z-q (2017) TGF-β in pancreatic cancer initiation and progression: two sides of the same coin. Cell & Bioscience 7: 39
Shevchenko I, Karakhanova S, Soltek S, Link J, Bayry J, Werner J, Umansky V, Bazhin A V (2013) Low-dose gemcitabine depletes regulatory T-cells and improves survival in the orthotopic Panc02 model of pancreatic cancer. Int J Cancer 133(1): 98-107
Sistigu A, Viaud S, Chaput N, Bracci L, Proietti E, Zitvogel L (2011) Immunomodulatory effects of cyclophosphamide and implementations for vaccine design. Seminars in immunopathology 33(4): 369-83
Speiser D E, Migliaccio M, Pittet M J, Valmori D, Lienard D, Lejeune F, Reichenbach P, Guillaume P, Luscher I, Cerottini J C, Romero P (2001) Human CD8(+) T-cells expressing HLA-D R and CD28 show telomerase activity and are distinct from cytolytic effector T-cells. Eur J Immunol 31(2): 459-66
Sun Z, Chen F, Meng F, Wei J, Liu B (2017) MHC class II restricted neoantigen: A promising target in tumor immunotherapy. Cancer letters
Thomas D A, Massague J (2005) TGF-beta directly targets cytotoxic T-cell functions during tumor evasion of immune surveillance. Cancer cell 8(5): 369-80
Topalian S L, Rosenberg S A (1987) Therapy of cancer using the adoptive transfer of activated killer cells and interleukin-2. Acta haematologica 78 Suppl 1: 75-6
Tran E, Ahmadzadeh M, Lu Y C, Gros A, Turcotte S, Robbins P F, Gartner J J, Zheng Z, Li Y F, Ray S, Wunderlich J R, Somerville R P, Rosenberg S A (2015) Immunogenicity of somatic mutations in human gastrointestinal cancers. Science 350(6266): 1387-90
Tran E, Robbins P F, Lu Y C, Prickett T D, Gartner J J, Jia L, Pasetto A, Zheng Z, Ray S, Groh E M, Kriley I R, Rosenberg S A (2016) T-Cell Transfer Therapy Targeting Mutant KRAS in Cancer. The New England journal of medicine 375(23): 2255-2262
Tran E, Turcotte S, Gros A, Robbins P F, Lu Y C, Dudley M E, Wunderlich J R, Somerville R P, Hogan K, Hinrichs C S, Parkhurst M R, Yang J C, Rosenberg S A (2014) Cancer immunotherapy based on mutation-specific CD4+ T-cells in a patient with epithelial cancer. Science 344(6184): 641-5
Tuller F, Holzer H, Schanda K, Aboulenein-Djamshidian F, Hoftberger R, Khalil M, Seifert-Held T, Leutmezer F, Berger T, Reindl M (2016) Characterization of the binding pattern of human aquaporin-4 autoantibodies in patients with neuromyelitis optica spectrum disorders. Journal of neuroinflammation 13(1): 176
Tureci O, Vormehr M, Diken M, Kreiter S, Huber C, Sahin U (2016) Targeting the Heterogeneity of Cancer with Individualized Neoepitope Vaccines. Clinical cancer research: an official journal of the American Association for Cancer Research 22(8): 1885-96
Valdez H, Smith K Y, Landay A, Connick E, Kuritzkes D R, Kessler H, Fox L, Spritzler J, Roe J, Lederman M B, Lederman H M, Evans T G, Heath-Chiozzi M, Lederman M M (2000) Response to immunization with recall and neoantigens after prolonged administration of an HIV-1 protease inhibitor-containing regimen. ACTG 375 team. AIDS Clinical Trials Group. Aids 14(1): 11-21
von Engelhardt J, Mack V, Sprengel R, Kavenstock N, Li K W, Stern-Bach Y, Smit A B, Seeburg P H, Monyer H (2010) CKAMP44: a brain-specific protein attenuating short-term synaptic plasticity in the dentate gyrus. Science 327(5972): 1518-22
Von Hoff D D, Ervin T, Arena F P, Chiorean E G, Infante J, Moore M, Seay T, Tjulandin S A, Ma W W, Saleh M N, Harris M, Reni M, Dowden S, Laheru D, Bahary N, Ramanathan R K, Tabernero J, Hidalgo M, Goldstein D, Van Cutsem E, Wei X, Iglesias J, Renschler M F (2013) Increased survival in pancreatic cancer with nab-paclitaxel plus gemcitabine. The New England journal of medicine 369(18): 1691-703
Von Hoff D D, Ramanathan R K, Borad M J, Laheru D A, Smith L S, Wood T E, Korn R L, Desai N, Trieu V, Iglesias J L, Zhang H, Soon-Shiong P, Shi T, Rajeshkumar N V, Maitra A, Hidalgo M (2011) Gemcitabine plus nab-paclitaxel is an active regimen in patients with advanced pancreatic cancer: a phase I/II trial. Journal of clinical oncology: official journal of the American Society of Clinical Oncology 29(34): 4548-54
Waddell N, Pajic M, Patch A M, Chang D K, Kassahn K S, Bailey P, Johns A L, Miller D, Nones K, Quek K, Quinn M C, Robertson A J, Fadlullah M Z, Bruxner T J, Christ A N, Harliwong I, Idrisoglu S, Manning S, Nourse C, Nourbakhsh E, Wani S, Wilson P J, Markham E, Cloonan N, Anderson M J, Fink J L, Holmes O, Kazakoff S H, Leonard C, Newell F, Poudel B, Song S, Taylor D, Waddell N, Wood S, Xu Q, Wu J, Pinese M, Cowley M J, Lee H C, Jones M D, Nagrial A M, Humphris J, Chantrill L A, Chin V, Steinmann A M, Mawson A, Humphrey E S, Colvin E K, Chou A, Scarlett C J, Pinho A V, Giry-Laterriere M, Rooman I, Samra J S, Kench J G, Pettitt J A, Merrett N D, Toon C, Epari K, Nguyen N Q, Barbour A, Zeps N, Jamieson N B, Graham J S, Niclou S P, Bjerkvig R, Grutzmann R, Aust D, Hruban R H, Maitra A, lacobuzio-Donahue C A, Wolfgang C L, Morgan R A, Lawlor R T, Corbo V, Bassi C, Falconi M, Zamboni G, Tortora G, Tempero M A, Australian Pancreatic Cancer Genome I, Gill A J, Eshleman J R, Pilarsky C, Scarpa A, Musgrove E A, Pearson J V, Biankin A V, Grimmond S M (2015) Whole genomes redefine the mutational landscape of pancreatic cancer. Nature 518(7540): 495-501 WHO (2014) World Cancer Report 2014 Lyon: International Agency for Research on Cancer, World Health Organisation.
Wu J, Yan Z, Li Z, Yan C, Lu S, Dong M, Yan N (2015) Structure of the voltage-gated calcium channel Cav1.1 complex. Science 350(6267): aad2395
Yang W, Shen N, Ye D Q, Liu Q, Zhang Y, Qian X X, Hirankarn N, Ying D, Pan H F, Mok C C, Chan T M, Wong R W, Lee K W, Mok M Y, Wong S N, Leung A M, Li X P, Avihingsanon Y, Wong C M, Lee T L, Ho M H, Lee P P, Chang Y K, Li P H, Li R J, Zhang L, Wong W H, Ng 10, Lau C S, Sham P C, Lau Y L, Asian Lupus Genetics C (2010) Genome-wide association study in Asian populations identifies variants in ETS1 and WDFY4 associated with systemic lupus erythematosus. PLoS genetics 6(2): e1000841
Zacharakis N, Chinnasamy H, Black M, Xu H, Lu Y-C, Zheng Z, Pasetto A, Langhan M, Shelton T, Prickett T, Gartner J, Jia L, Trebska-McGowan K, Somerville R P, Robbins P F, Rosenberg S A, Goff S L, Feldman S A (2018) Immune recognition of somatic mutations leading to complete durable regression in metastatic breast cancer. Nature medicine 24(6): 724-730
Zhao J, Cao Y, Lei Z, Yang Z, Zhang B, Huang B (2010) Selective depletion of CD4+CD25+Foxp3+ regulatory T-cells by low-dose cyclophosphamide is explained by reduced intracellular ATP levels. Cancer research 70(12): 4850-8

1. A method for producing a T-cell product containing tumor uber reactive immune cells (TURICS) comprising the steps of
a) providing a body sample containing T-cells of a patient;
b) optionally isolating the T-cells from the body sample;
c) stimulating the T-cells in vitro in the presence of a cytokine cocktail of the cytokines interleukin 2 (IL-2), interleukin 15 (IL-15) and interleukin 21 (IL-21) and a stimulating peptide or a group of stimulating peptides;
d) determining a reactivity factor in the T-cell sample, wherein said reactivity factor is indicative for the presence of T-cells targeting the stimulating peptide or at least one peptide of the group of stimulating peptides;
e) in case the reactivity factor is positive, identifying the T-cell sample as a tumor reactive T-cell sample; otherwise identifying the T-cell sample as a non-reactive T-cell sample;
f) culturing the non-reactive sample in vitro in the presence of the cytokine cocktail of IL-2, IL-15 and IL-21 and either one of autologous tumor cells or the stimulating peptide or the group of stimulating peptides to form a T-cell product;
g) optionally stimulating the T-cell product in vitro in the presence of the cytokine cocktail of IL-2, IL-15 and IL-21 and the stimulating peptide or the group of stimulating peptides;
h) determining the reactivity factor in the T-cell product; and
i) in case the reactivity factor is positive selecting the T-cell product as a T-cell product containing TURICS.

2. The method according to claim 1, wherein the group of stimulating peptides consists of up to 20 different stimulating peptides, preferably up to 10 different stimulating peptides, more preferably up to five different stimulating peptides.

3. The method according to claim 1 or 2, wherein the stimulating peptides are mutated or non-mutated tumor-specific peptides, wherein the mutated tumor specific peptides contain an amino acid sequence with a mutation found in tumor cells of the patient but not in cells of healthy tissue of the patient.

4. The method according to claim 3, wherein the mutation is located in the middle of the peptide.

5. The method according to any of the previous claims, wherein the stimulating peptides have a length in the range from 5 to 31 amino acids, preferably 7 to 25 amino acids, more preferably 9 to 21 amino acids.

6. The method according to any of the previous claims, wherein the stimulation of the T-cells and/or the T-cell product is performed on 10² to 10⁸ cells, preferably on 10³ to 10⁶ cells, more preferably on 10⁴ to 10⁵ cells.

7. The method according to any of the previous claims, wherein the T-cells and/or the T-cell product are stimulated for 1 hour to 10 days, preferably for 3 hours to 5 days, more preferably 1 day to 3 days.

8. The method of any of the previous claims, wherein the non-reactive T-cell sample is cultured for 1 to 10 days, preferably for 3 to 9 days, more preferably for 6 to 8 days.

9. The method according to any of the previous claims, wherein in steps c) and g) the stimulating peptide or each peptide of the group of stimulating peptides is present in a concentration of from 1 μg/10⁵ cells to 1 mg/10⁵ cells, preferably in a concentration of 1 ng/10⁵ cells to 100 μg/10⁵ cells, more preferably in a concentration of from 1 μg/10⁵ cells to 10 μg/10⁵ cells.

10. The method of any of the previous claims, wherein the non-reactive T-cell sample and the autologous tumor cells are cultured in a ratio ranging from of 1000:1 to 1:1000, preferably in a ratio ranging from of 10:1 to 1:10, more preferably in a ratio of 7:1 to 3:1.

11. The method according to any of the previous claims, wherein the reactivity factor is selected from T-cell proliferation, cytokine production, cytotoxicity, e.g. killing of the cells of the diseased body sample, degranulation, in particular defined by CD107a positivity, maturation and or differentiation, in particular defined by the combination of CD45RA and CCR7, expression of a T-cell activation marker in particular selected from CD25, CD56, CD69 of MHC class II molecules; an exhaustion and/or activation markers selected from Foxp3, LAG-3, TIM-3, 4-1BB, PD-1, CD127 (IL-7R), the IL-21 receptor or T-cell signaling, in particular selected from the zeta chain phosphorylation, preferably, the reactivity factor is the IFNγ concentration and the reactivity factor is positive if the concentration of IFNγ is above a predefined IFNγ threshold.

12. The method according to any of the previous claims, wherein
steps c) and d) are additionally carried out with a comparative peptide or a group of comparative peptides as the stimulating peptide or the group of stimulating peptides, wherein the comparative peptide(s) contain(s) the non-mutated sequence corresponding to the mutated tumor specific peptide sequence, when a mutated tumor specific peptides was used as stimulating peptide, and/or
steps g) and h) are additionally carried out with a comparative peptide or a group of comparative peptides as the stimulating peptide or the group of stimulating peptides, wherein the comparative peptide(s) contain(s) the non-mutated sequence corresponding to the mutated tumor specific peptide sequence, when a mutated tumor specific peptides was used as stimulating peptide, and wherein the T-cell product is deselected in case the reactivity factor for stimulating the T-cell product with the comparative peptide or the group of comparative peptides is equal to or higher than the reactivity factor for stimulating the T-cell product with the mutated tumor specific peptide or the group of tumor specific peptides.

13. The method according to any of the previous claims, wherein the body sample is not a tumor a sample, preferably the tumor sample is selected from whole blood, serum, plasma, urine, tears, sperm, saliva, synovial fluid, umbilical cord, placenta tissue, bone marrow, exhaled air, lavage material, such as bronchoalveolar lavage, cerebrospinal fluid (CSF), primary, secondary lymphoid tissues, samples from the gut lumen, samples from peritoneal cavity, transplanted material, transplanted cells, transplanted tissue(s) or organ(s).

14. The method according to claim 12, wherein each comparative peptide is applied in a concentration similar to that of the corresponding tumor-specific peptide.

15. A composition containing at least one T-cell product with TURICs for use in treatment of a cancer patient, comprising performing the method according to any one of claims 1 to 14 to obtain a T-cell product with TURICs and administering the T-cell product with TURICs to the patient.
